(19) United States
(12) Patent Application Publication
(10) Pub. No.: US 2024/0067603 A1
(43) Pub. Date: Feb. 29, 2024

(54) CUBANYL BIGUANIDE COMPOUNDS

(71) Applicants: REGENTS OF THE UNIVERSITY OF MINNESOTA, MINNEAPOLIS, MN (US); ICAHN SCHOOL OF MEDICINE AT MOUNT SINAI, New York, NY (US)

(72) Inventors: David A. POTTER, MINNEAPOLIS, MN (US); Zhijun GUO, MINNEAPOLIS, MN (US); Gunda GEORG, MINNEAPOLIS, MN (US); Kwon Ho HONG, MINNEAPOLIS, MN (US); Jianxun LEI, MINNEAPOLIS, MN (US); Elizabeth AMBROSE, MINNEAPOLIS, MN (US); Kris WHITE, New York, NY (US)

(21) Appl. No.: 18/258,076

(22) PCT Filed: Dec. 16, 2021
(86) PCT No.: PCT/US2021/063848

§ 371 Date: Jun. 16, 2023

Related U.S. Application Data
(60) Provisional application No. 63/126,389, filed on Dec. 16, 2020.

(51) Int. Cl. C07C 279/26; A61P 31/14

(52) U.S. Cl. CPC C07C 279/26; A61P 31/14; C07C 2603/90

(57) ABSTRACT

Biguanide compounds of formula

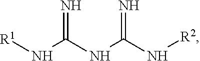

where R¹ comprises a cubanyl-substituted alkyl and R² comprises a substituted or unsubstituted alkyl, and their use in the treatment of cancers and viral infections are described.

### CROSS-REFERENCE TO RELATED APPLICATIONS

The present application claims the benefit of priority to U.S. Patent Application Ser. No. 63/126,389, filed Dec. 16, 2020, the contents of which is incorporated by reference in its entirety.

### STATEMENT REGARDING FEDERALLY SPONSORED RESEARCH

This invention was made with government support under CA113570 awarded by the National Institutes of Health and under W81XWH1910099 awarded by the U.S. Department of Defense. The government has certain rights in the invention.

### BACKGROUND OF THE INVENTION

Cancer cell-intrinsic cytochromes P450 (CYP) monooxygenases promote tumor progression. In estrogen positive HER2 negative breast cancer (ER+HER2−) breast cancer cells, CYP3A4 promotes tumor growth, in part, through epoxyeicosatrienoic acid (EET) biosynthesis. The biguanide diabetes drug metformin is currently being studied in breast cancer clinical trials. While metformin and biguanides, buformin and phenformin, exhibit inhibitory activity against breast cancer they lack potency and their mechanisms of action are unclear. Accordingly, there is a need for more potent agents, including biguanides that treat breast cancer including estrogen positive breast cancer including ER+HER2− breast cancer.

### BRIEF SUMMARY OF THE INVENTION

Disclosed herein are biguanide compounds and their use for the treatment of cancers or viral infections. In one embodiment, the biguanide compound is a cubanyl biguanide compound of formula

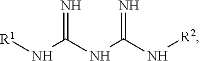

or a pharmaceutically acceptable salt thereof, wherein R¹ comprises a cubanyl-substituted alkyl and R² comprises a substituted or unsubstituted alkyl.

Another aspect of the invention provides for pharmaceutical composition comprising any of the compounds described herein. In some embodiments, the pharmaceutical composition is formulated for subcutaneous administration.

Another aspect of the invention provides for a method for treating a subject in need of a treatment for a cancer. The method may comprise administering an effective amount of any of the compounds described herein. Suitably, the cancer is a breast cancer, such as an ER+ breast cancer, a HER2+ breast cancer, or a triple negative breast cancer. In some embodiments, the method further comprises co-administering and effective amount of an immunotherapy, such as an antibody or check point inhibitor.

Another aspect of the invention provides for a method for inhibiting a cancer cell-intrinsic CYP monooxygenase. The method may comprise contacting the CYP with any of the compounds described herein.

Another aspect of the invention is a method for inhibiting the growth, inhibiting proliferation, or killing of a cell. The method may comprise contacting the cell with any of the compounds described herein.

Another aspect of the invention provides for a method for inhibiting biosynthesis of an epoxyeicosatrienoic acid (EET) within a cell. The method may comprise contacting the cell with any of the compounds described herein.

Another aspect of the invention provides for activating a 5′ adenosine monophosphate-activated protein kinase (AMPK). The method may comprise contacting a cell with any of the compounds described herein.

Another aspect of the invention provides for a method for inhibiting expression of ERα. The method may comprise contacting a cell with any of the compounds described herein.

Another aspect of the invention provides for a method for inhibiting nuclear translocation. The method may comprise contacting a cell with any of the compounds described herein.

Another aspect of the invention provides for a method of inactivating or inhibiting replication of a virus. The method may comprise contacting a virus infected cell with any of the compounds described herein. In some embodiments, the virus is a coronavirus such as a SARS virus, e.g., SARS-CoV-2.

Another aspect of the invention provides for a method for treating a subject in need of a treatment for a viral infection. The method may comprise administering an effective amount of any of the compounded described herein. In some embodiments, the virus is a coronavirus such as a SARS virus, e.g., SARS-CoV-2.

Another aspect of the invention provides for a method for improving the bioavailability of a biguanide compound. The method may comprise subcutaneously administering the biguanide compound to a subject in need of the biguanide compound. In some embodiments, the subject is in need of a treatment for cancer, such as a breast cancer. In some embodiments, the subject is in need of a treatment for viral infection, such as a coronavirus infection.

In some embodiments, the compound is

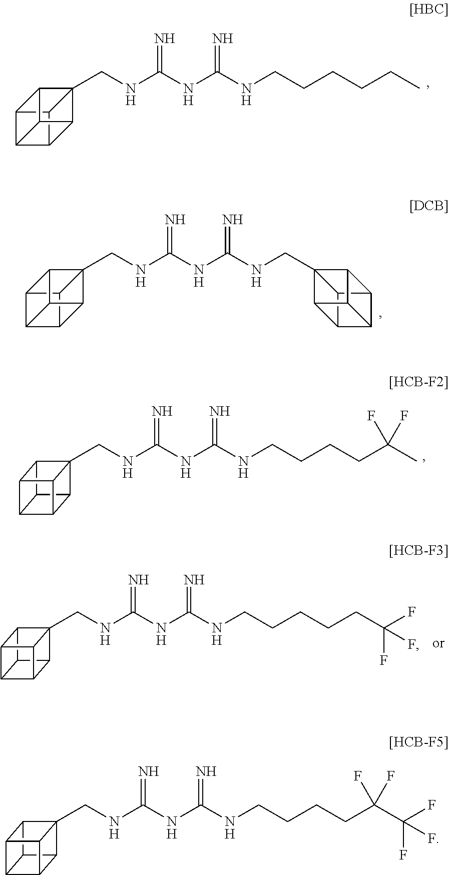 [SMILES: CCCCCCNC(=N)NC(=N)NCC12C3C4C5C3C1C5C42 ; N=C(NCC12C3C4C5C3C1C5C42)NC(=N)NCC12C3C4C5C3C1C5C42 ; CC(F)(F)CCCCNC(=N)NC(=N)NCC12C3C4C5C3C1C5C42 ; N=C(NCCCCCC(F)(F)F)NC(=N)NCC12C3C4C5C3C1C5C42 ; "or" ; N=C(NCCCCC(F)(F)C(F)(F)F)NC(=N)NCC12C3C4C5C3C1C5C42]

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Effects of HCB on CYP3A4 Supersome™ mediated EET biosynthesis. A 50 ul reaction in a 1.5 ml microcentrifuge tube contains the following components: arachidonic acid (60 μM), NDAPH (2 mM), DMSO or drug, CYP3A4 Supersome™ (1 μM) in potassium phosphate buffer (pH=7.4). The reaction was initiated by the addition of Supersome™ to the reaction mixture. After incubation at 37° C. for 30 minutes, 50 ul of acetonitrile spiked with 13C-EET internal standards was added and vortexed vigorously to terminate the reaction. Microcentrifuge tubes were centrifuged at 16,000×rpm for 10 min to precipitate proteins and other insoluble debris and 80 ul supernatant was transferred into HPLC vials and analyzed by an LC-MS as described previously (1). Results are expressed as mean±S.D. (n=3). IC50s were determined by curve fitting as 4.7±0.2, 26.5±1.9 and 64.8±6.5 uM for (±)14,15-EET, (±)11,12-EET and (±)8,9-EET, respectively.

FIG. 2. Effects of DCB and HCB on CYP3A4 Supersome™ mediated EET biosynthesis. A 0.5 ml reaction in a 10 ml glass test tube contains the following components: arachidonic acid (30 mM), NDAPH (2 mM), DMSO or drug solution (0.5 μl), CYP3A4 Supersome™ (80 nM) and 450.5 ul potassium phosphate buffer (pH=7.4). The reaction was initiated by the addition of Supersome™ to the rest. After incubation at 37° C. for 30 minutes, 0.5 ml of dichloromethane was added and vortexed vigorously to terminate the reaction. Organic phase and aqueous phase were separated by centrifugation at 3,000×rpm for 10 minutes. Two hundred microliters of the organic phase was withdrawn and evaporated to dryness. The residues were reconstituted with 20 ul methanol containing ¹³C-EET internal standards and analyzed by an LC-MS as described previously (1). Results are expressed as mean±S.D. (n=3, * indicates a statistically significant difference from the control).

FIGS. 3A-3B. Effects of DCB (FIG. 3B) and HCB (FIG. 3A) on MCF-7 cells respiration. MCF-7 cells were seeded at 25,000 cells/well into a 96-well XF96 cell microplate. Growth media was replaced with 150 μL metabolism assay media the next day and cells were incubated CO₂ free for 1 hour before the measurement of oxygen consumption rate (OCR) every seven minutes by a Seahorse XFe analyzer. Drugs were injected into the media after the fifth measurement (indicated by time point A and the dashed line). Results are reported as mean of percent of base OCR (OCR of the fifth measurement)±S.D. (n=12 for DMSO and 16 for treatment groups).

FIG. 4A. Effect of HCB on mitochondrial cross membrane potential Δψ in MCF-7 cells. MCF-7 cells were seeded at 20,000 cells/well into a clear bottom black 96 well microplate. After treatments were added for 2 hours (FCCP control for 10 minutes), TMRE (tetramethylrhodamine, ethyl ester, Ex/Em 549/575) was added to a final concentration of 400 nM and incubation continued for additional 30 minutes. Media was removed and cells washed twice with PBS and 100 ul of PBS was added in the end. TMRE fluorescence was then measured with a plate reader. Results are reported as mean fluorescence intensity±S.D. (n=8, * indicates statistically significant difference from the DMSO control).

FIG. 4B. Effect of DCB and HCB on mitochondrial trans-membrane potential Δψ in MCF-7 cells. MCF-7 cells were seeded at 20,000 cells/well into a clear bottom black 96 well microplate. After treatments were added for 2 hours (FCCP control for 10 minutes), TMRE (tetramethylrhodamine, ethyl ester, Ex/Em 549/575) was added to a final concentration of 400 nM and incubation continued for additional 30 minutes. Media was removed and cells washed twice with PBS and 100 ul of PBS was added at the end. TMRE fluorescence was then measured with a plate reader. Results are reported as mean fluorescence intensity±S.D. (n=8, * indicates statistically significant difference from the DMSO control).

FIG. 5. Mitochondrial swelling by HCB treatment in MCF-7 cells. MCF-7 cells were seeded at 30,000 cells/well into a chambered coverslip and treated with HCB or DMSO for 2 hours. JC-1 potential sensitive dye was added to a final concentration of 20 μM and incubation continued for an additional half an hour. Cell was then washed and imaged with a confocal microscope. Active mitochondria are stained yellow to orange. White arrows indicate swollen mitochondria.

FIG. 6. HCB (10 μM) treatment for 2 hours decreases nuclear translocation of ERα in MCF-7 cells. A. representative images (Blue: nuclei; Red: ERα) from an experiment. B. Percentage change of nuclear ERα density normalized to nuclear DNA stained by Hoechst. MCF-7 cells (1.5×10⁴ cells/well) were seeded on uSlide (8 wells, Ibidi, cat #80826) and cultured with complete culture medium (MEM medium with Earle's salts & L-glutamine) containing 10% FBS, 10 mM HEPES, 2 mM glutamine and 100 U penicillin and 100 U streptomycin for almost 70 hours. Then the cells were starved in serum, phenol red and antibiotics free MEM medium for almost 16 hours, followed with HCB (10 μM) or vehicle (0.01% DMSO) treatment for 2 hours in starvation medium. Then the cells were fixed, permeabilized and probed with monoclonal antibody against ERα (Abcam, Cat #16660, primary antibody) and Texas Red-conjugated goat anti rabbit IgG (Thermo Fisher Scientific, Cat #T-6391, secondary antibody). Nuclei were stained with Hoechst dye (Thermo Scientific, Cat #62249). The nuclear ERαas were analyzed with confocal microscopy. Four sites per well were selected randomly for collecting the image data. The density of fluorescence was measured by ImageJ. With auto thresholding in the blue (Hoechst dye, nuclei) channel, individual nuclear area was selected as a region of interest (ROI). Intensities of lights in each ROI were measured in the blue channel (nuclei) and red (Texas red, estrogen receptor α) channel. The amount of nuclear ERα was presented with the ratio of density of ERα in nuclear region to density of nucleus (Hoechst dye). Results normalized to control are expressed as means±S.D. (n=7). Comparison was performed with Student-t test (unpaired, two-tailed). * indicates statistically significant difference (p<0.05).

FIG. 7. Effect of HCB on MCF-7 AMPKα phosphorylation (Thr172). MCF-7 cells were grown in complete media to 70% confluency and treated with 10 μM HCB. Cells were harvested at varied time (0-24 hr) and total proteins were extracted and analyzed by Western blotting. For 0% serum experiment, complete media was replaced with serum, phenol red and antibiotics free media 16 hours before treatment. Quantitation results are expressed as mean±S.D. (n=3, * indicates statistically significant difference, p<0.05). MCF-7 cells were grown in complete media (10% serum) to 80% confluence. Left panel: HCB was added to a final concentration of 10 μM in complete media; Right panel: complete media was replaced with serum free media and serum starved for 16 hours before HCB was added to a final concentration of 10 μM. At indicated time points after the addition of HCB, cells were harvested and analyzed by Western blotting. AMPK activation was reported as fractional Thr172 phosphorylation±S.D. (n=3, * indicates statistically significant difference from control).

FIG. 8. Effects of HCB on the proliferation of MCF-7 cells. MCF-7 cells were seeded in 96 well plates and treated with drugs at varied concentration for 24 hours. MTT assay was performed to determine remaining viable cells in each well. Results are represented as mean±S.D. (n=7). IC₅₀ was determined by curve fitting. IC₅₀ for DCB is 22.1±1.7 μM and HCB 8.4±1.2 μM.

FIG. 9. Results of cell proliferation assay.

FIG. 10. Results of cell proliferation assay.

FIG. 11. Shows Vero cells (African green monkey kidney cells), remdesivir was compared with HCB and HBB.

FIG. 12. Shows 293T human embryonic kidney cells over-expressing the ACE2 receptor for SARS-CoV-2. In the 293T/ACE2 assay, remdesivir was compared with HCB and HBB.

FIG. 13. Biguanides inhibit NF-κB in epithelia. A. Latent NF-κB. AMPK inhibits IKKβ. EETs open the nuclear pore complex, but NF-κB remains anchored in the cytoplasm. AMPK keeps IKKβ in check. PPARγ plays a protective role and is a control for our studies. B. Activation of JAK2/NF-κB by TNα a or viral proteins. AMPK low, allowing IKKβ to promote loss of lκBα. C. Open nuclear pore, but impaired entry of NF-κB due to inhibition of IKKβ by HBB activation of AMPK, regardless of ROS production, which would otherwise activate IKKβ. HBB also inhibits EET induced STAT3 (JAK2 TBD).

FIG. 14. Pharmacokinetic profile of HBB.

FIG. 15. Pharmacokinetic profile of HCB.

FIG. 16. HCB induces a low-to-high spin transition in CYP3A4.

FIG. 17. Spectra of CYP3A4-ND with HCB added from 0 to 218 μM

FIG. 18. Titration of CYP3A4-ND with HCB (data from FIG. 17)

FIG. 19. Left—1 OH/4 OH ratio of products of MDZ hydroxylation. Right—rates of MDZ hydroxylation (1 OH+4 OH, total product). Both panels show the results for MDZ hydroxylation in the absence of HCB (black triangles) and in the presence of indicated concentrations of HCB.

FIG. 20. Tolerance of dose escalation.

FIG. 21. Tolerance of HBB dosing above 12 mg/kg.

FIG. 22. Tolerance of daily HBB dosing at 12 mg/kg.

FIG. 23. Tumor volume progression comparing HBB vs. PBS and maintenance of weight in HBB-treated mice.

### DETAILED DESCRIPTION OF THE INVENTION

Disclosed herein are cubanyl biguanide compounds and methods of using biguanide compounds for treating cancers, such as ER+HER2− breast cancer, ER+HER2+ breast cancer, triple negative breast cancer, and viral infections, such as SARS-CoV-2 viral infections. As demonstrated in the examples that follow, the presently disclosed compounds demonstrated unexpectedly high anticancer and antiviral activity.

### Compounds

One aspect of the technology provides for the use of biguanide compounds. Biguanide is a compound of formula

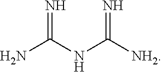

A variety of biguanide compounds may be prepared by optionally substituting one or more of the terminal hydrogen with a substituent to prepare a compound of formula

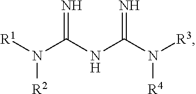

where R¹, R², R³, and R⁴ are independently selected from H, C₁₋₁₂ alkyl, C₂₋₁₂ alkenyl, C₂₋₁₂ alkynyl, —O-(C₁₋₁₂)alkyl, —O-(C₂₋₁₂)alkenyl, —O-(C₂₋₁₂ )alkynyl, —OH, C₃₋₈ carbocycle, a 5-10 membered heteroaryl or aryl, wherein any alkyl, alkenyl, alkynyl, -alkyl, —O-alkenyl, —O-alkynyl, carbocycle, heteroaryl, or aryl is optionally substituted with one or more (e.g., 1, 2, 3, 4, 5 or more) substituents. Substituents may be selected from halo, hydroxide, —O-(C₁₋₆)alkyl, —C(═O)O-(C₁₋₆)alkyl, C₃₋₈ carbocycle, or a 5-10 membered heteroaryl or aryl, wherein any carbocycle, heteroaryl, or aryl may be optionally substituted with one or more (e.g., 1, 2, 3, 4, 5 or more) groups selected from C₁₋₆ alkyl, —OH, halo and —O-(C₁₋₆)alkyl. Exemplary biguanide compounds are provided for in U.S. Pat. Nos. 9,416,098, 9,993,446, 10,272,055, and 10,376,480.

Cancer cell-intrinsic CYP monooxygenases promote tumor progression. In ER+HER2−, breast cancer cells, CYP3A4 promotes tumor growth, in part, through epoxyeicosatrienoic acid (EET) biosynthesis. Metformin, N,N-dimethylbiguanide, binds to the active site heme of CYP3A4, thereby inhibiting EET biosynthesis and metformin analog N1-hexyl-N5-benzyl-biguanide (HBB)

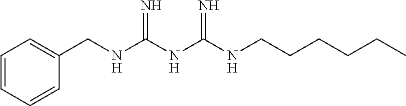 [SMILES: CCCCCCNC(=N)NC(=N)NCc1ccccc1]

is a 100-fold more potent. HBB specifically inhibits CYP3A4 arachidonic acid (AA) epoxygenase activity in breast cancer cells, suppresses the growth of breast cancer cell lines (IC₅₀=3-30 μM), and inhibits growth of the MCF-7 ER+ mammary tumor model, similar to CYP3A4 gene silencing.

The present disclosure improves on the potency and pharmacological properties of HBB and other biguanides compounds by incorporation of a bridged, polycyclic carbocycle. The bridged, polycyclic carbocycle is a cubanyl moiety. Cubane, pentacyclo[4.2.0.02,5.03,8.04,7] octane, comprises eight carbon atoms arranged into the shape of a cube with one hydrogen atom attached to each of the carbon atoms. Cubanes are biologically stable, lipophilic platforms on which a wide choice of substituents in a variety of well-defined spatial relationships can be installed. A cubanyl moiety comprises eight carbon atoms arranged into the shape of a cube where one of the carbon atoms serves as the point of attachment to a biguanide compound. The cubanyl moiety may be optionally substituted. The cubanyl moiety may be a substituent of one or more of R¹, R², R³, and R⁴.

In some embodiments, the compound is a cubanyl biguanide compounds of formula

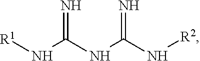

or a pharmaceutically acceptable salt thereof, where at least one of R¹ and R² comprises a cubanyl moiety. In some embodiments, both or R¹ and R² comprise a cubanyl moiety.

In some embodiments, R¹ comprises a cubanyl-substituted C₁₋₁₂ alkyl and R² comprises a substituted or unsubstituted C₁₋₁₂ alkyl. In certain embodiments, both of R¹ and R² comprise a cubanyl-substituted alkyl. Suitably, the alkyl group linking the cubanyl moiety may be a C₁₋₄ alkyl, including without limitation methyl, ethyl, propyl, or butyl. In particular embodiments, R¹ and, optionally, R² may be

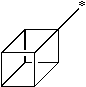 [SMILES: *C12C3C4C5C3C1C5C42]

where * indicated the point of attachment to a nitrogen of the biguanide compound.

In some embodiments, R² comprises an unsubstituted C₁₋₁₂ alkyl. Suitably, R² is methyl, ethyl, propyl, butyl, pentyl, hexyl, heptyl, octyl, nonyl, decyl, or the like. In some embodiments, R² is hexyl.

In some embodiments, R² is substituted C₁₋₁₂ alkyl. In particular embodiments, the alkyl is substituted with a carbocycle, such as a cubanyl moiety described above. In other embodiments, the alkyl is halo substituted, such as with one or more fluorine atoms.

Exemplary cubanyl biguanide compounds include

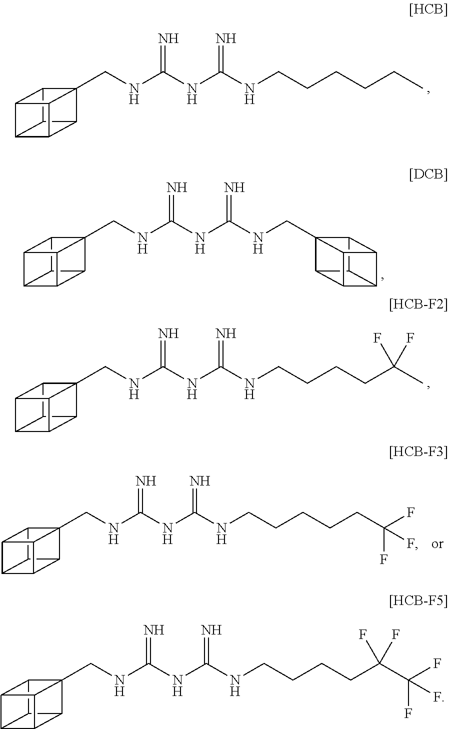 [SMILES: CCCCCCNC(=N)NC(=N)NCC12C3C4C5C3C1C5C42 ; N=C(NCC12C3C4C5C3C1C5C42)NC(=N)NCC12C3C4C5C3C1C5C42 ; CC(F)(F)CCCCNC(=N)NC(=N)NCC12C3C4C5C3C1C5C42 ; N=C(NCCCCCC(F)(F)F)NC(=N)NCC12C3C4C5C3C1C5C42 ; N=C(NCCCCC(F)(F)C(F)(F)F)NC(=N)NCC12C3C4C5C3C1C5C42]

In some embodiments, the cubanyl biguanide compound is a pharmaceutically acceptable salt. In some embodiments, the salt of the cubanyl biguanide compound is a mesylate salt.

As used herein, an asterisk “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C₁₋₁₂ alkyl, C₁₋₁₀ alkyl, and C₁₋₆ alkyl, respectively.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like

The term “heteroalkyl” as used herein refers to an “alkyl” group in which at least one carbon atom has been replaced with a heteroatom (e.g., an O, N, or S atom). One type of heteroalkyl group is an “alkoxyl” group

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂₋₁₂ alkenyl, C₂₋₁₀ alkenyl, and C₂₋₆ alkenyl, respectively

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂₋₁₂ alkynyl, C₂₋₁₀ alkynyl, and C₂₋₆ alkynyl, respectively

The term “cycloalkyl” or “carbocycle” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The terms “cubane” and “cubanyl” are art-recognized and refer to hydrocarbon molecules containing eight carbon atoms arranged at the corners of a cube.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cₓ₋ₓ nomenclature where x is an integer specifying the number of ring atoms. For example, a C₃₋₇ heterocyclyl group refers to a saturated or partially unsaturated 3- to 7-membered ring structure containing one to four heteroatoms, such as nitrogen, oxygen, and sulfur. The designation “C₃₋₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

An “epoxide” is a cyclic ether with a three-atom ring typically include two carbon atoms and whose shape approximates an isosceles triangle. Epoxides can be formed by oxidation of a double bound where the carbon atoms of the double bond form an epoxide with an oxygen atom.

The term “carbonyl” as used herein refers to the radical —C(O)—.

The term “carboxamide” as used herein refers to the radical —C(O)NRR′, where R and R′ may be the same or different. Rand R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —R¹C(O)N(R²)—, —R¹C(O)N(R²)R³—, —C(O)N R²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.)

### Pharmaceutical Compositions

The compounds utilized in the methods disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended-release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s). In some embodiments, the pharmaceutical compositions described herein are administered subcutaneously.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that treats cancer activity may be administered as a single compound or in combination with another compound that treats cancer or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like. In addition, the methods disclosed herein may be practiced using solvate forms of the compounds or salts, esters, and/or amides, thereof. Solvate forms may include ethanol solvates, hydrates, and the like.

### Methods of Cancer Treatment

The compounds disclosed herein may be used for methods of treatment. In some embodiments, the compounds may be used for methods of treatment of cancer. The methods of treatment may include the administration of a therapeutically effective amount of the compounds described herein to a subject. The compounds may be any of the cubanyl biguanid compounds described herein, including, without limitation, HCB, DCB, HCB-F2, HCB-F3, HCB-F5, or any combination thereof.

Treating the disease or infection includes, without limitation, alleviating one or more clinical indications, inhibiting tumor growth, decreasing tumor size, reducing the severity of one or more clinical indications of the cancer, diminishing the extent of the condition, stabilizing the subject's disease (i.e., not worsening), delay or slowing, halting, or reversing the disease or infection and bringing about partial or complete remission of the disease or cancer. Treating the disease or infection also includes prolonging survival by days, weeks, months, or years as compared to prognosis if treated according to standard medical practice not incorporating treatment with the compounds or a pharmaceutically acceptable salt thereof.

In some embodiments, the cancer is breast cancer. Breast cancer is a heterogeneous disease that can be classified using a variety of clinical and pathological features. Classification may help in prognostication and targeting of treatment to those most likely to benefit. Currently, estrogen receptor (ER) status and human epidermal growth factor receptor-2 (HER2) status are used as predictive markers to select specific therapies. Immunohistochemical (IHC) has been used to classify tumors. Generally, a hierarchical classification is used, with luminal and nonluminal tumors defined as those tumors that express either ER or progesterone receptor (PR) and those that do not. The luminal and nonluminal groups can then be further subdivided according to HER2-expression status to generate four subtypes, and these four subtypes can each be categorized according to whether or not they express a basal marker yielding a total of eight subtypes.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with cubanyl biguanide compounds. For example, a “subject in need of treatment” may include a subject having a cell proliferative disease, disorder, or condition such as cancer, including breast cancer.

As used herein the term “effective amount” refers to the amount or dose of the compound, upon single or multiple dose administration to the subject or one that provides the desired effect. The disclosed methods may include administering an effective amount of the disclosed compounds (e.g., as present in a pharmaceutical composition) for treating a cell proliferative disease or disorder, such as a cancer or, more particularly, breast cancer.

In some embodiments, the breast cancer is an ER+ breast cancer. In some embodiments, the ER+ breast cancer is an ER+HER2− breast cancer or ER+HER2+ breast cancer.

In some embodiments, the breast cancer is an HER2+ breast cancer. In some embodiments, the HER2+ breast cancer is an ER+HER2+ breast cancer of a ER−HER2+ breast cancer.

In some embodiments, the breast cancer is a triple negative breast cancer. Triple negative breast cancers refer to tumors that do not express ER, PR, or HER2.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

In some embodiments, the compound is co-administered with an immunotherapy. The immunotherepay may be administered before, during or after administration of the compound. In some embodiments, the immunotherapy is capable of binding to or inhibiting a cell surface receptor associated with an immunological response. Suitable the immunotherapy may be an antibody or a checkpoint inhibitor. In some embodiments, the immunotherapy may be capable of binding to or inhibiting a marker indicative of T-cell exhaustion. Suitably, the immunotherapy may bind to or inhibit one or more of one or more of PD-1, PDL-1, CTLA4, LAG3, or TIGIT. Exemplary immunotherapies include, without limitation, pembrolizumab, nivolumab, atezolizumab, avelumab, cemiplimab, ipilimumab, REGN3767, or a combination thereof. Methods for inhibiting the growth or proliferation of or for killing a cell

The compounds described herein may be used for inhibiting the growth or proliferation of or for killing a cell. Suitably, the method comprises contacting the cell with an effective amount of any of the compounds described herein, including, without limitation, HCB, DCB, HCB-F2, HCB-F3, HCB-F5, or any combination thereof. The method may be practiced in vitro, ex vivo, or in vivo.

The cell may be a cancer cell. In some embodiments, the cell is a breast cancer cell.

In some embodiments, the breast cancer cell is an ER+ breast cancer cell. In some embodiments, the ER+ breast cancer cell is an ER+HER2− breast cancer cell or ER+HER2+ breast cancer cell.

In some embodiments, the breast cancer cell is an HER2+ breast cancer cell. In some embodiments, the HER2+ breast cancer cell is an ER+HER2+ breast cancer cell of a ER−HER2+ breast cancer cell.

In some embodiments, the breast cancer is a triple negative breast cancer cell. Methods for inhibiting a cancer cell-intrinsic CYP monooxygenase

The cubanyl biguanide compounds may be used for inhibiting a cancer cell-intrinsic CYP monooxygenase. Suitably, the method comprises contacting a CYP with an effective amount of any of the cubanyl biguanide compounds described herein. The method may be practiced in vitro, ex vivo, or in vivo. As disclosed herein, the term “inhibiting” may include blocking enzyme activity and/or reducing enzyme activity by a statistically significant amount. Suitably, enzyme activity may be reduced by 10%, 20%, 30%, 40%, 50%, 60%, 70%, 80%, 90, or more.

“Cytochromes P450” or “CYPs” are a superfamily of enzymes containing heme as a cofactor that function as monooxygenases. In mammals, these proteins oxidize steroids, fatty acids, and xenobiotics, and are important for the clearance of various compounds, as well as for hormone synthesis and breakdown. In plants, these proteins are important for the biosynthesis of defensive compounds, fatty acids, and hormones. CYPs are, in general, the terminal oxidase enzymes in electron transfer chains, broadly categorized as P450-containing systems.

A “cancer cell-intrinsic CYP” is a CYP that is present or originates from within the cell.

In some embodiments, the CYP that is inhibited upon contact with the compounds described herein is Cytochrome P450 3A4 (CYP3A4). CYP3A4 is mainly found in the liver and in the intestine. CYP3A4 promotes the growth of various types of cancer cell line cultures. CYP3A4 has been shown to promote breast cancer progression. CYP3A4 metabolizes arachidonic acid to produce epoxyeicosatrienoic acids (EETs).

In some embodiments, the CYP that is inhibited upon contact with the compounds described herein is Cytochrome P450 2C8 (CYP2C8). CYP2C8 metabolizes long-chain polyunsaturated fatty acids. CYP2C8 metabolizes arachidonic acid to epoxyeicosatrienoic acids (EETs). CYP2C8 metabolizes linoleic acid to 9, 10-epoxy octadecaenoic acids (also known as vernolic acid) and 12, 13-epoxy-octadecaenoic (also known as coronaric acid). CYP2C8 metabolizes docosahexaenoic acid to various epoxydocosapentaenoic acids (also known as EDPs). CYP2C8 metabolizes eicosapentaenoic acid to various epoxyeicosatetraenoic acids (also known as EEQs).

### Methods for Inhibiting Biosynthesis of EET

The cubanyl biguanide compounds may be used for inhibiting biosynthesis of an epoxyeicosatrienoic acid (EET) in a cell. Suitably, the method comprises contacting the cell with an effective amount of any of the compounds described herein. The method may be practiced in vitro, ex vivo, or in vivo.

Epoxyeicosatrienoic acids (EETs) have various regioisomers such as 5,6-EET (5,6-epoxy-8Z, 11Z,14Z-eicosatrienoic acid). 8,9-EET (8,9-epoxy-5Z,11Z,14Z-eicosatiienoic acid). 11,12-EET (11,12-epoxy-5Z,8Z,14Z-eicosatrienoic acid), or 15-EET (14,15-epoxy-5Z,8Z,11Z-eicosatrienoic acid). CYPs generally produce both R/S enantiomers at each former double bond position; for example, some CYPs metabolize arachidonic acid to a mixture of 14R,15S-EET and 14S,15R-EET. In some embodiments, the cubanyl biguanide compounds inhibit the biosynthesis of (±)14,15-EET.

The cell may be a cancer cell. In some embodiments, the cell is a breast cancer cell.

In some embodiments, the breast cancer cell is an ER+ breast cancer cell. In some embodiments, the ER+ breast cancer cell is an ER+HER2− breast cancer cell or ER+HER2+ breast cancer cell.

In some embodiments, the breast cancer cell is an HER2+ breast cancer cell. In some embodiments, the HER2+ breast cancer cell is an ER+HER2+ breast cancer cell of a ER−HER2+ breast cancer cell.

In some embodiments, the breast cancer is a triple negative breast cancer cell.

### Methods for Activating AMPK

The compounds described herein may be used for activating a 5′ adenosine monophosphate-activated protein kinase (AMPK) in a cell. Suitably, the method comprising contacting a cell with the cubanyl biguanide compounds described herein. In some embodiments, the AMPK is AMPKα. In some embodiments, the Thr172 of AMPKα is phosphorylated. The method may be practiced in vitro, ex vivo, or in vivo.

The cell may be a cancer cell. In some embodiments, the cell is a breast cancer cell.

In some embodiments, the breast cancer cell is an ER+ breast cancer cell. In some embodiments, the ER+ breast cancer cell is an ER+HER2− breast cancer cell or ER+HER2+ breast cancer cell.

In some embodiments, the breast cancer cell is an HER2+ breast cancer cell. In some embodiments, the HER2+ breast cancer cell is an ER+HER2+ breast cancer cell of a ER−HER2+ breast cancer cell.

In some embodiments, the breast cancer is a triple negative breast cancer cell.

### Methods for Inhibiting Expression of ERα

The compounds described herein may be used for inhibiting expression of estrogen receptor alpha (ERα). Suitably, the method comprising contacting a cell with the disclosed herein. The method may be practiced in vitro, ex vivo, or in vivo.

ERα, also known as nuclear receptor subfamily 3, group A, member 1(NR3A1), is a main type of estrogen receptor, a nuclear receptor that is activated by estrogen. ERα plays a role in the physiological development and function of a variety of organ systems to varying degrees, including the reproductive, central nervous, skeletal, and cardiovascular systems. Accordingly, ERα is widely expressed throughout the body, including the uterus and ovary, male reproductive organs, mammary gland, bone, heart, hypothalamus, pituitary gland, liver, lung, kidney, spleen, and adipose tissue.

The cell may be a cancer cell. In some embodiments, the cell is a breast cancer cell. In some embodiments, the breast cancer cell is an ER+ breast cancer cell. In some embodiments, the ER+ breast cancer cell is an ER+HER2− breast cancer cell or ER+HER2+ breast cancer cell.

### Methods for Inhibiting Nuclear Translocation

The compounds described herein may be used for inhibiting nuclear translocation in a cell. Suitably, the method comprises contacting the cell with an effective amount of any of the compounds described herein. The method may be practiced in vitro, ex vivo, or in vivo.

Nuclear translocation is a subcellular process in which activated cytoplasmic proteins are transported into the cell nucleus as part of a signal transduction pathway to modify cell function in response to a signaling event or condition.

In some embodiments, the nuclear translocation of ERα is inhibited.

The cell may be a cancer cell. In some embodiments, the cell is a breast cancer cell. In some embodiments, the breast cancer cell is an ER+ breast cancer cell. In some embodiments, the ER+ breast cancer cell is an ER+HER2− breast cancer cell or ER+HER2+ breast cancer cell.

Although CYP3A4 synthesizes EETs, how EETs may promote tumor growth is unknown. Advantageously, the Inventors found that HBB inhibits nuclear transit of wor alpha (ERα) and 70kD FITC-dextran and metformin inhibits nuclear pore complex (NPC) function. Although the mechanism is unknown it is believed that CYP-derived EETs function as second messengers that regulate nuclear translocation of ERα in breast cancer by promoting permeability of the NPC. This implies that biguanide drugs suppress nuclear translocation of the ERα through inhibition of CYP-derived EETs that serve as second messengers to open the NPC.

### Methods of Treatment for a Viral Infection

The compounds disclosed herein may be used in methods of treatment for a viral infection. The methods of treatment may include the administration of a therapeutically effective amount of the compounds described herein to a subject.

In some embodiments, the viral infection is an infection by a coronavirus. Coronaviruses are a large family of viruses that usually cause mild to moderate upper-respiratory tract illnesses, like the common cold. Exemplary coronaviruses include SARS-CoV-2, which causes coronavirus disease 2019 (COVID-19): middle East respiratory syndrome (MERS) coronavirus (MERS-CoV), which causes MERS; or SARS-CoV, which causes severe acute respiratory syndrome (SARS). In some embodiments, the virus is a mTOR dependent virus or an oxidative phosphorylation dependent virus.

As used herein, the terms “treat” and “treating” refers to therapeutic measures, wherein the object is to slow down (lessen) an undesired physiological change or pathological disorder resulting from a disease or infection as described herein. Treating the disease or infection includes, without limitation, alleviating one or more clinical indications, decreasing inflammation, reducing the severity of one or more clinical indications of the disease or infection, diminishing the extent of the condition, stabilizing the subject's disease or infection (i.e., not worsening), delay or slowing, halting, or reversing the disease or infection and bringing about partial or complete remission of the disease or infection. Treating the disease or infection also includes prolonging survival by days, weeks, months, or years as compared to prognosis if treated according to standard medical practice not incorporating treatment with the compounds or a pharmaceutically acceptable salt thereof. For example, symptoms of COVID-19 include, but are not limited to, for example, fever, cough, shortness of breath or difficulty breathing, chills, repeated shaking with chills, muscle pain, headache, sore throat, new loss of taste or smell, trouble breathing, persistent pain or pressure in the chest, new confusion or inability to arouse, bluish lips or face, among others.

Subjects in need of treatment can include those already having or diagnosed with a disease or infection as described herein as well as those prone to, likely to develop, or suspected of having a disease or infection as described herein. Pre-treating or preventing a disease or infection according to a method of the present invention includes initiating the administration of a therapeutic (e.g., the cubanyl biguanide compounds or a pharmaceutically acceptable salt thereof) at a time prior to the appearance or existence of the disease or infection, or prior to the exposure of a subject to factors known to induce the disease or infection. Pre-treating the disorder is particularly applicable to subjects at risk of having or acquiring the disease. As used herein, the terms “prevent” and “preventing” refer to prophylactic or preventive measures intended to inhibit undesirable physiological changes or the development of a disorder or condition resulting from the disease or infection. In exemplary embodiments, preventing the disease or infection comprises initiating the administration of a therapeutic (e.g., the compounds or a pharmaceutically acceptable salt thereof) at a time prior to the appearance or existence of the disease or infection such that the disease or infection, or its symptoms, pathological features, consequences, or adverse effects do not occur. In such cases, a method of the invention for preventing the disease or infection comprises administering the cubanyl biguanide compounds or a pharmaceutically acceptable salt thereof to a subject in need thereof prior to exposure of the subject to factors that influence the development of the disease or infection. In some embodiments, the subject is a human and the administration of the compounds or a pharmaceutically acceptable salt thereof is intended to prevent advancement of COVID-19 following exposure of the human subject to SARS-CoV-2 or following diagnosis of SARS-CoV-2 infection in the subject. In some embodiments, the subject is s subject that has tested positive for SARS-CoV-2, or a subject that has been exposed to a person who tested positive for SARS-CoV-2.

### Method of Inactivating a Virus or Inhibiting Viral Replication

The compounds described herein may be used for inactivating or inhibiting replication of a virus in a cell. Suitably, the method comprising contacting a virus infected cell with the compounds described herein.

There are currently no strategies to inhibit NF-kB-mediated pulmonary inflammation in coronavirus infection. Severe acute respiratory syndrome coronavirus 2 (SARS-CoV-2) causes Pulmonary infection in corona virus disease 2019 (COVID-19). SARS-Cov-2 triggers a local cytokine storm, or excessive production of inflammatory mediators resulting in tissue damage, leading to respiratory failure and enhanced viral replication. Cytokine storm is mediated, in part, by JAK2 activation of the pleiotropic transcription factor NF-kB, a well-known promoter of innate and adaptive immunity and inducer of inflammatory mediators. While JAK2 inhibitors have been developed, they may be insufficient to block NF-kB activation and coronavirus replication. NF-kB inhibition has been proposed as a target for suppression of inflammation in coronavirus respiratory infection, because a strategy to lower NF-kB activity improves survival in a mouse model. Nonetheless, there no approved drugs specifically designed to inhibit NF-kB function, perhaps related to the pleiotropic nature of NF-kB in immunity.

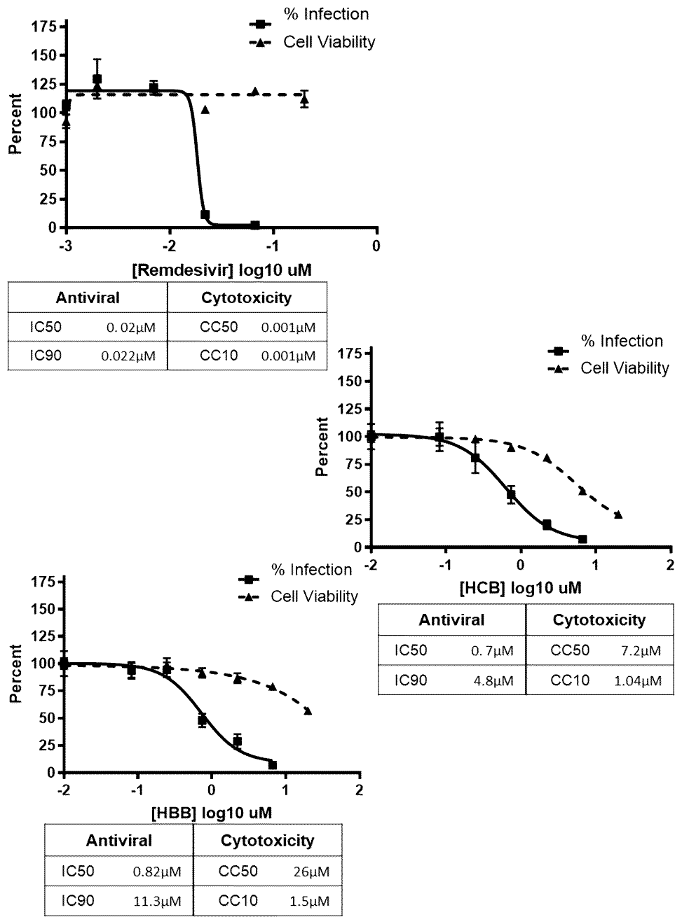

The heme containing mitochondrial cytochrome P450 (CYP) arachidonic acid epoxygenase enzymes provides for potent lung protective agents. Epoxides of arachidonic acid (EETs) inhibit NF-kB signaling, in part, through inhibition of IKKβ, activity of which is required for nuclear transit of NF-kB. Advantageously, the Inventors have found that EETs inhibit AMPK, potentially relieving inhibition of IKKβ, but they also suppress ROS, the net effect being inhibition of NF-κB nuclear transit (FIG. 13). The compounds described herein inhibit EET biosynthesis and the electron transport chain (ETC), thereby weakly promoting ROS. The compounds described herein also activate AMPK thereby inhibiting IKKβ and nuclear transit of NF-κB. Without wishing to be bound to theory, it is believed that NF-κB requires 2 signals to enter the nucleus during viral pathogenesis, activation of JAK2/IKKβ and ROS (FIG. 13).

HBB (20 uM) inhibits nuclear transit of NF-kB, has acceptable toxicity in mice and no observed effect on splenic or lymph node CD4 or CD8 T cells; there are differential epithelial vs. T cell effects. Metformin and the novel biguanides inhibit the JAK/STAT pathway as well. ROS may be irrelevant in the model and that potent inhibition of IKKβ by biguanide activated AMPK may be sufficient to block nuclear transit of NF-κB. Metformin, HBB, and HCB may be used to inhibit NF-κB-mediated inflammation promoted by coronavirus activation of JAK2 and IKKβ, thereby ameliorating cytokine release syndrome, which contributes to viral pathogenesis.

In some embodiments, the cell is infected with a virus. In some embodiments, the virus is a coronavirus. The coronavirus may be, but is not limited to, SARS-CoV-2, MERS-CoV, or SARS-CoV.

In some embodiments, the biguanide is co-administered with an antiviral agent. The antiviral agent may be administered before, during or after administration of the bigunide. Exemplary immunotherapies include, without limitation, remdesivir.

### Methods for Increasing the Bioavailability of a Biguanide Compound

Another aspect of the invention provides for a method for improving the bioavailability of a biguanide compound. The method may comprise subcutaneously administering the biguanide compound to a subject in need of the biguanide compound. Subcutaneous administration of the compound results in a larger area under the curve (AUC), lower toxicity, and a higher maximum tolerated dose (MTD) than when the compound is administered by another administration method.

In some embodiments, the bioavailability of the biguanide, compound as measured by the AUC, is larger when the biguanide compound is administered subcutaneously than when administered orally, intravenously, or intraperitoneally.

In some embodiments, the biguanide is less toxic to the subject when the biguanide compound is administered subcutaneously than when administered orally, intravenously, or intraperitoneally.

In some embodiments, the maximum tolerated dose (MTD) is larger when the biguanide compound is administered subcutaneously than when administered orally, intravenously, or intraperitoneally.

In some embodiments, the subject is in need of a treatment for cancer, such as a breast cancer. In some embodiments, the subject is in need of a treatment for viral infection, such as a coronavirus infection.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example A. Biguanide Cubanyl Compounds of the Present Disclosure

CYP3A4 promotes breast cancer progression. It has been shown that the biguanide compound hexyl-benzyl-biguanide (HBB) is an inhibitor of CYP3A4 and have demonstrated that metformin and other biguanides inhibit breast cancer, in part, by inhibiting CYP3A4 and other arachidonic acid (AA) epoxygenase enzymes [Cell Chem Biol. 2017 Oct. 19; 24(10):1259-1275].

The present invention improves on the potency and pharmacological properties of HBB. Cubanes have the same size of the phenyl moiety, but are aliphatic rather than aromatic, and may thereby exhibit higher affinity binding to the CYP3A4 target, lower electrophysiological risk of heart arrhythmias, and decreased CYP-mediated metabolism through phenyl ring hydroxylation. Fluorine substitution, at or near positions, where HBB is most vulnerable to CYP-mediated hydroxylation may improve compound stability and lessen metabolic breakdown.

To this end, compounds comprising a methylene-cubanyl had substantially improved Surflex-dock scores for CYP3A4 binding. (Table 1) Docking of cubane and fluorine substitution of HBB-like structures led to a series of new structures expected to be more potent inhibitors of CYP3A4 AA epoxygenase activity based on prediction of higher affinity binding (Table 1).

These compounds were also found to have a decreased or equal predicted QPlogHERG compared to HBB, which has an acceptable value (Table 2) with the cubanes exhibiting significantly improved HERG signals compared to HBB. Higher schools correlated with decreased electrophysiological risk.

Fluorine atom substitution may also improve blood brain barrier penetration to treat metastatic disease in the brain as evidenced by a greater QPPMDCK score (Table 3), predicting better transit of the blood brain barrier. Higher scores are correlated with a higher likelihood of crossing the blood brain barrier.

Table 4 shows by QPPCaco scores that the general permeability of these compounds to enter cells and pass the gut blood barrier to be good for all. Higher scores correlate with higher gut absorption.

Based on these finding, the cubane and fluorinated derivatives are expected to outperform HBB as cancer drugs.

In order to calculate docking scores, compounds were sketched in SYBYL-X 2.1 (Certara, L.P.) and prepared using the ligand preparation tool with Surflex for searching as the preparation protocol. A structure of CYP3A4 (PBD ID: 5G5J) was prepared using the structure preparation tool: all waters were deleted, metformin was extracted as a ligand substructure, termini were set to their charged states, all hydrogens were added, atom types were assigned using AMBER7 FF02, charges were added using AMBER7 FF02 as the biopolymer force field and Gasteiger-Hückel as the ligand/metal force field, the iron atom's charge was specified as 3.0, sidechain amides were fixed, and biopolymer hydrogens were minimized using current charges and Tripos as the force field. Docking experiments were conducted using Surflex-Dock and CScore in SYBYL-X with Surflex-Dock GeomX (SFXC) as the docking mode. In order to calculate predicted physicochemical property values, compounds were sketched in Maestro 11.9.011 (Schrödinger, Inc.), prepared using LigPrep, and analyzed using QikProp. Important predictions include predicted IC50 values for the blockage of HERG K+ channels (QPlogHERG); predicted non-active, apparent Caco-2 cell permeabilities as a model for the gut-blood barrier (QPPCaco); and predicted non-active, apparent MDCK cell permeabilities as a model for the blood-brain barrier (QPPMDCK). All modeling studies were carried out on Minnesota Supercomputing Institute workstations running CentOS Linux Release 6.10 (Final).

TABLE 1
Surflex-Dock score of biguanide compounds.

| Compound | Surflex-Dock Score |
|---|---|
| HBB | 7.9316 |
| HCB | 8.0905 |
| HCB-F2 | 8.4437 |
| HCB-F3 | 8.0330 |
| HCB-F5 | 7.9813 |

TABLE 2
QPlog HERG scores indicated electrophysiological risk..

| Compound | Surflex-Dock Score |
|---|---|
| HBB | −5.492 |
| HCB | −4.742 |
| HCB-F2 | −4.729 |
| HCB-F3 | −5.057 |
| HCB-F5 | −4.815 |

TABLE 3
QPPMDCK scores for predicted MDCK cell uptake.

| Compound | Surflex-Dock Score |
|---|---|
| HBB | 287.625 |
| HCB | 416.545 |
| HCB-F2 | 1038.116 |
| HCB-F3 | 1691.370 |
| HCB-F5 | 4037.525 |

TABLE 4
QPPCaco scores for predicted Caco2 cell uptake.

| Compound | Surflex-Dock Score |
|---|---|
| HBB | 605.48 |
| HCB | 623.787 |
| HCB-F2 | 648.150 |
| HCB-F3 | 521.623 |
| HCB-F5 | 653.579 |

### Example B. Cubanyl Biguanides Inhibit Cytochrome P450 Driven Breast Cancer Mitochondrial Metabolism

Cancer cell-intrinsic CYP monooxygenases promote tumor progression through promotion of the electron transport chain (ETC), in part, through biosynthesis of epoxyeicosatrienoic acids (EETs). Metformin and its more potent analog N1-hexyl-N5-benzyl-biguanide (HBB) bind the active site heme of CYP3A4 and inhibit CYP3A4-mediated EET biosynthesis.

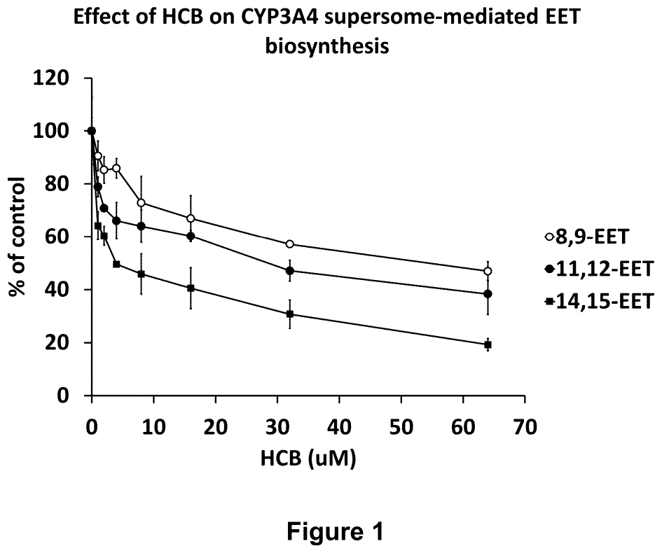

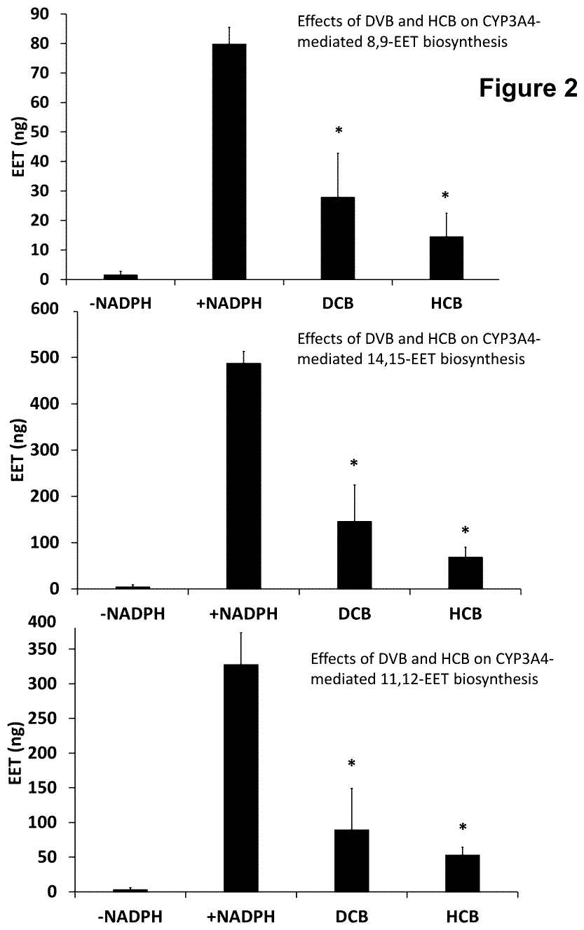

Compared to HBB and metformin, and as shown in FIGS. 1 and 2, HCB more potently and specifically inhibited CYP3A4 epoxygenase activity and was selective for inhibition of biosynthesis of the cancer promoting EET regioisomer, (±)-14,15-EET (Kᵢ=4.7 μM for HCB vs. 9 μM for HBB vs. 5 mM for metformin). HCB exhibits specificity for inhibition CYP3A4-mediated arachidonic acid epoxygenase biosynthesis of the most potent cancer promoting EET regioisomer, (±)14,15-EET, with an IC₅₀ of 3.8 uM, compared to an IC₅₀ of 9.5 uM for HBB inhibition of the same activity. HCB is therefore ˜2.5 fold more potent for inhibiting biosynthesis of (±)14,15-EET.

The HCB IC₅₀ values for CYP3A4 biosynthesis of the (±)8,9-EET and (±)11,12-EET regioisomers are 28 and 56 uM. HCB is also CYP3A4 specific. HCB inhibition of the CYP2C8-mediated arachidonic acid epoxygenase biosynthesis of (±)14,15-EET is 42 uM, indicating 10-fold lower inhibition of this activity. The HCB IC₅₀ values for CYP2C8 biosynthesis of the (±)8,9-EET and (±)11,12-EET regioisomers are 31 and 52 uM.

In summary, HCB is 2.5-fold more potent for inhibition of (±)14,15-EET biosynthesis by CYP3A4 and is more selective for CYP3A4, because it exhibits 10-fold lower inhibition of CYP2C8. CYP3A4 is specifically increased in ER-positive/HER2-negative breast cancer, growth of which is driven by the (±)14,15-EET regioisomer and we hypothesize that the more potent activity of HCB, compared to HBB, in ER-positive/HER2-negative breast cancer is driven, in part, but the selectivity of HCB for CYP3A4 and the inhibition of biosynthesis of (±)14,15-EET regioisomer.

This specificity, which was unexpected, means that HCB will have fewer off target effects on the cardiovascular system, hypertension and cardiac and vascular smooth muscle injury, which are ameliorated by 11,12-EET. There is also less effect on autoimmunity, because inflammatory processes are inhibited by 11,12-EET. Furthermore, HBB class biguanides do not inhibit CYP2J2, which is cardioprotective.

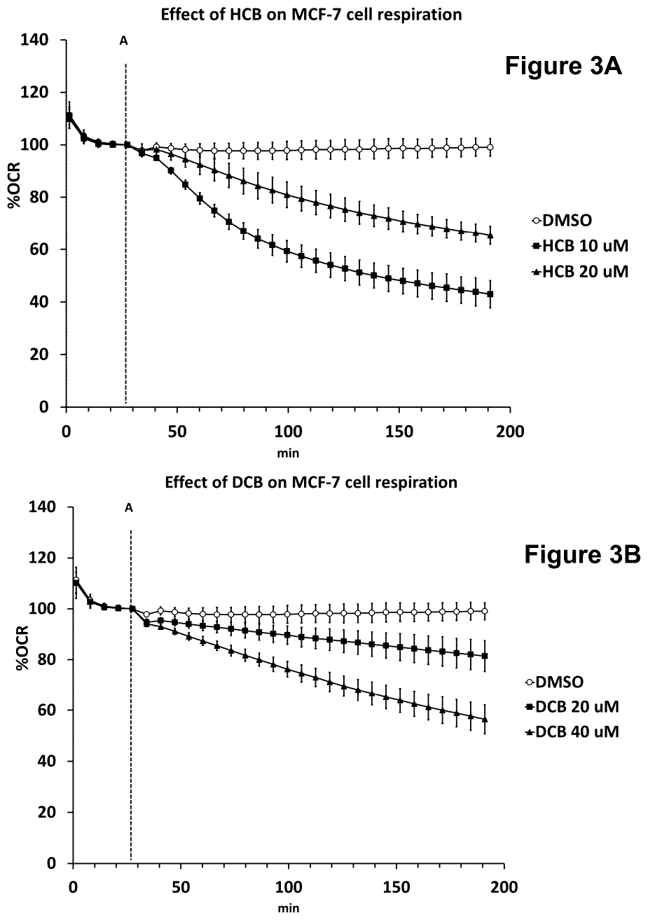

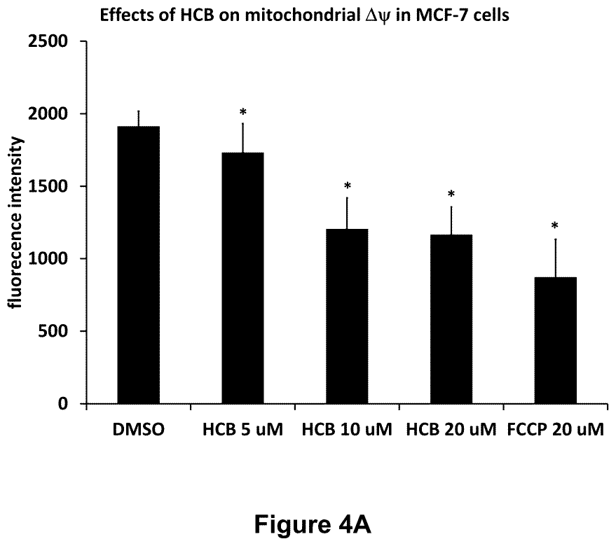

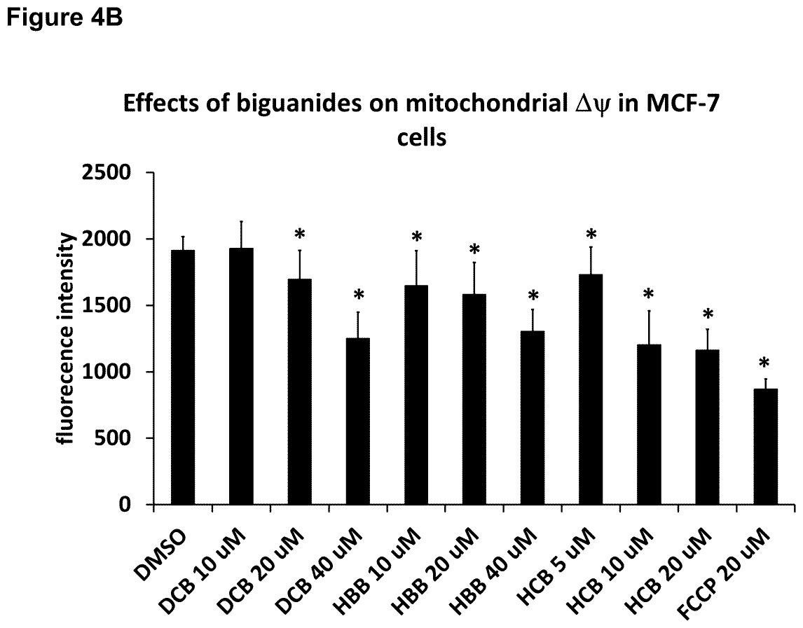

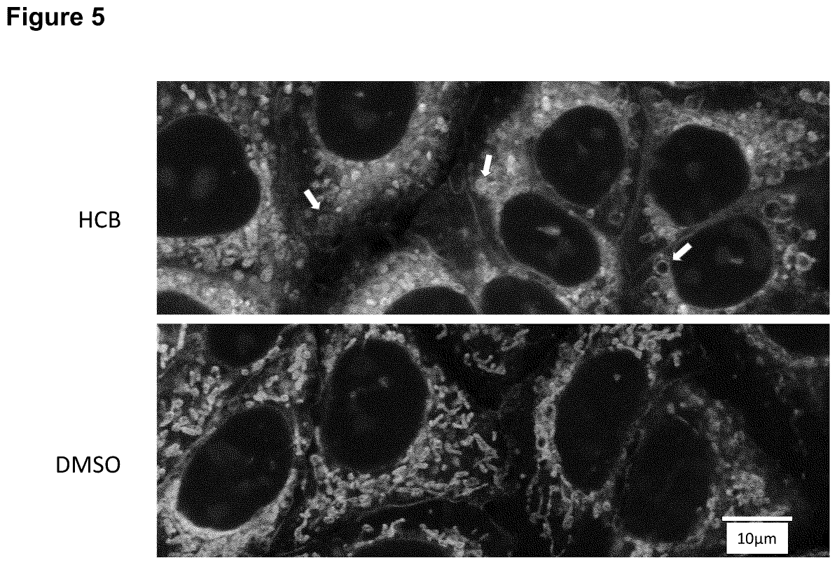

FIGS. 3A-3B shows that HCB (10 μM) inhibited oxidative phosphorylation, which is regulated in part by (±)-14,15-EET, inhibited the mitochondrial membrane potential, and caused loss of mitochondrial tubule networks (See FIGS. 4A-4B and 5).

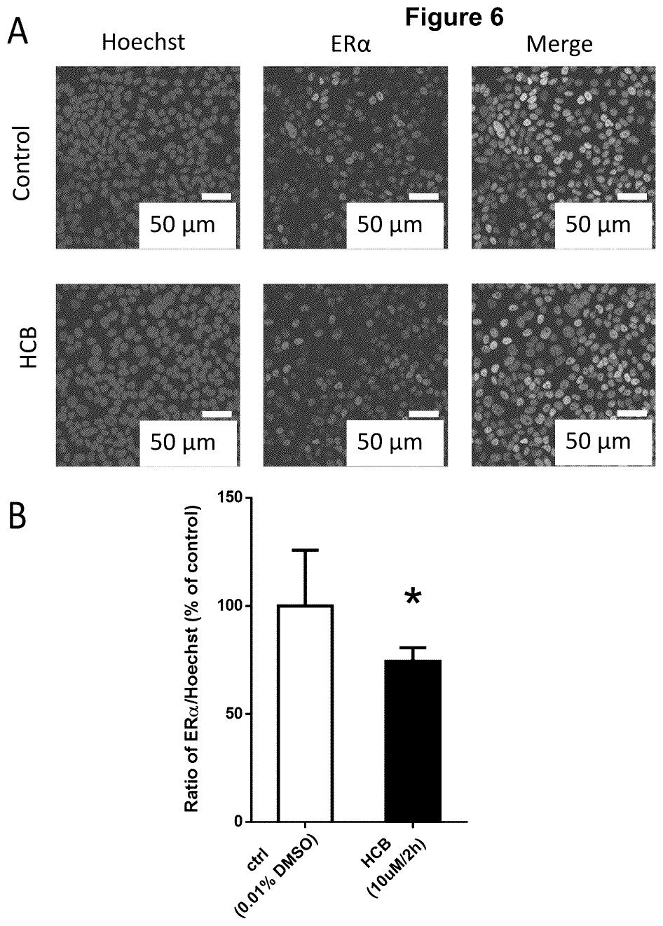

FIG. 6 shows HCB in 10 μM can significantly decrease the nuclear translocation of estrogen receptor (ER-α) in MCF-7 cells, while HBB exhibits similar effect in higher dose (20 uM), demonstrating that HCB is more potent than HBB. The nuclear transit of the estrogen receptor-α, tumor promoting processes driven, in part, by (±)-14,15-EET.

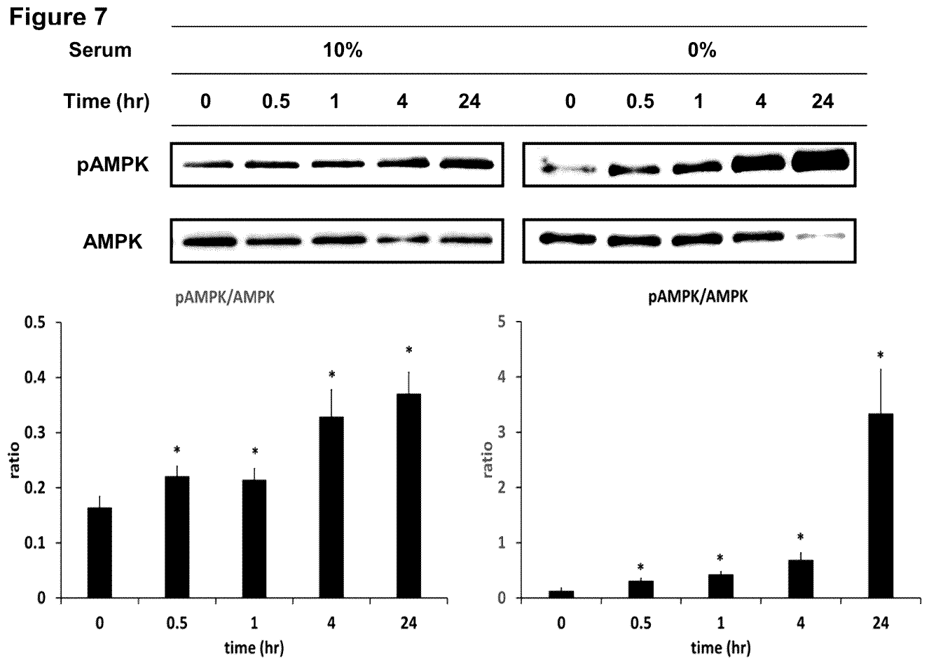

In cells, with and without serum, HCB (10 μM) exhibited potent activation of AMPK phosphorylation (See FIG. 7).

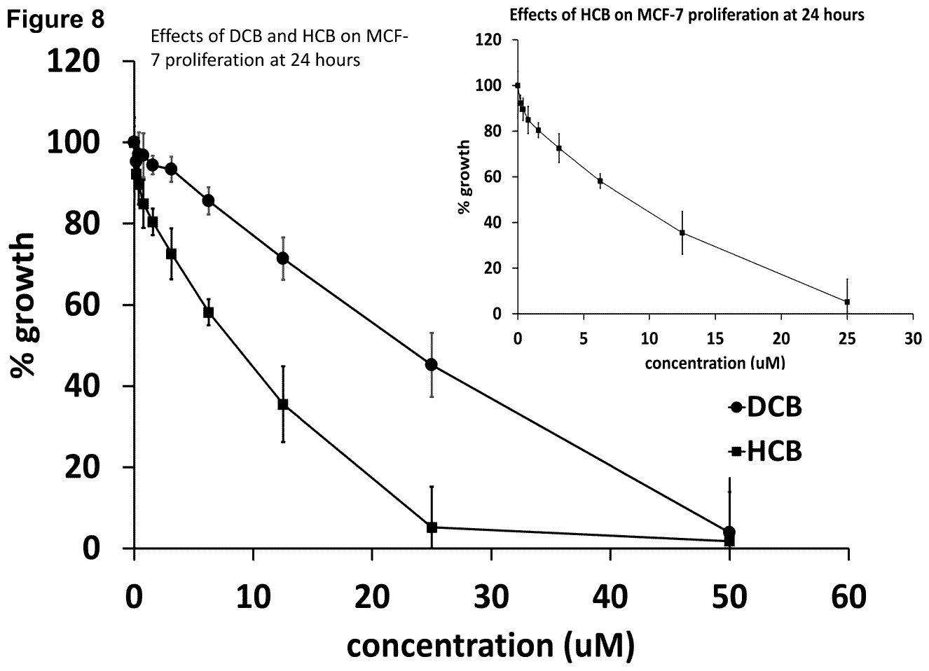

FIG. 8 show that HCB inhibit MCF-7 cells. IC₅₀ was determined by curve fitting. IC₅₀ for DCB is 22.1±1.7 μM and HCB 8.4±1.2 μM. Table 5 shows inhibition of mammary carcinoma cell lines by HCB and HBB.

HCB-F3 and HCB-F2 were also evaluated for their ability to inhibit MCF-7 cells. IC₅₀ was determined to be 13.7 and 13.5 μM, respectively.

TABLE 5
Mammary carcinoma cell lines and their inhibition by HCB and HBB

| | | | | | | OCR Inhibition | |
|---|---|---|---|---|---|---|---|
| | | | | HCB | HBB | [HCB] (uM) v | PD-1/PD- |
| | | | HER2 | IC50 | IC50 | vs. control | L1 blockade |
| Cell line | Strain | Histology | Status | (uM) | (uM) | (P < 0.05) | sensitive |
| mm001 | C57BL/6 | Basal | TBD | 12.1 ± | 26.8 ± | 10 | TBD |
| | | | | 0.05 | 0.5 | | |
| mm006 | C57BL/6 | Basal | TBD | 16.9 ± | 38.5 ± | 20 | TBD |
| | | | | 0.6 | 0.3 | | |
| EO771 | C57BL/6 | Basal | HER2− | TBD | 21.7 ± | 20 | TBD |
| | | | | | 0.15 | | |
| MCF-7 | human | ERα+ | HER2− | 8.4 ± | 22.2 ± | 10 | NA |
| | | | | 1.2 | 1.7 | | |
| T47D | human | ERα+ | HER2− | TBD | 22 ± | TBD | NA |
| | | | | | 4.2 | | |
| ZR75 | human | ERα+ | HER2− | TBD | TBD | TBD | |
| BT474 | human | ERα+ | HER2+ | 10.9 ± | 23.2 ± | | |
| | | | | 1.3 | 1.8 | | |
| MDA- | human | Basal | HER2− | 15.4 ± | 18.5 ± | TBD | NA |
| MB-231 | | | | 1.9 | 1.1 | | |

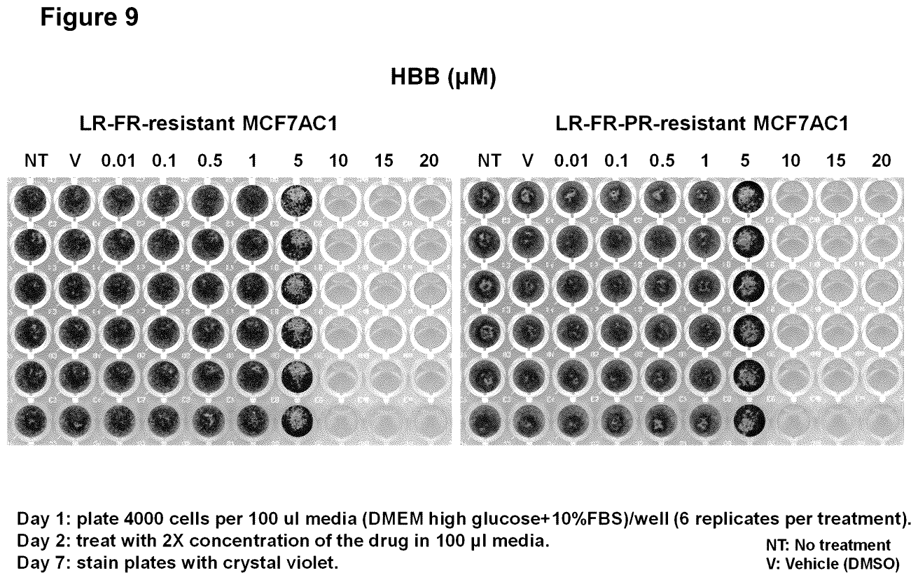

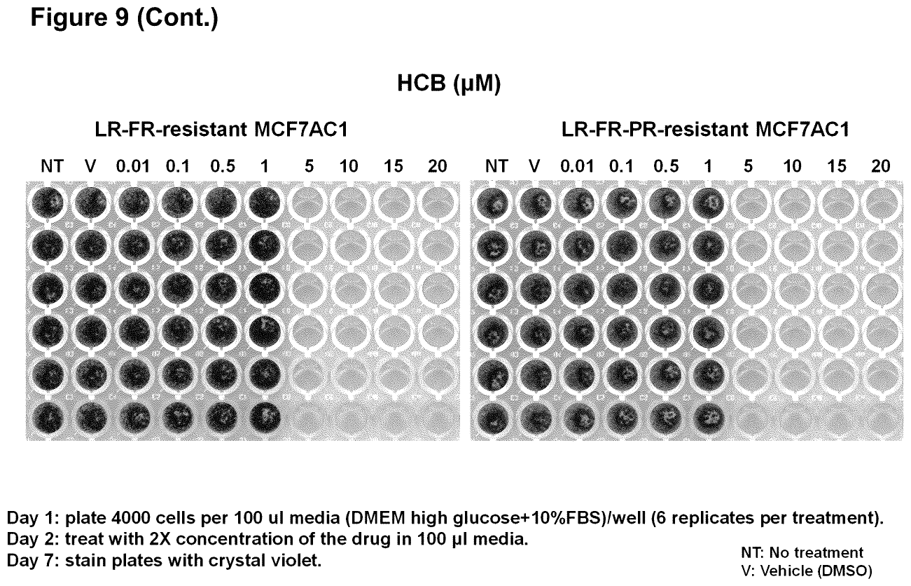

FIGS. 9 and 10 show that the letrozole- and fulvestrant-resistant MCF7AC1 (LR,FR-MCF7AC1) cells are resistant to letrozole and fulvestrant co-treatments and are capable of growth in the presence in 1 μM concentration of each drug (HCB and HBB) in phenol-red DMEM/F12+10% FBS media containing antibiotics. The letrozole, fulvestrant- and palbociclib-resistant MCF7AC1 (LR,FR,PR-MCF7AC1) cells are resistant to letrozole, fulvestrant and palbociclib cotreatments and are capable of growth in the presence in 1 μM concentration of each drug (HCB and HBB) in phenol-red DMEM/F12+10% FBS media containing antibiotics.

Bioisosteric cubanyl substitution of the aromatic ring of HBB therefore resulted in more potent inhibition of CYP epoxygenase enzymes as well as downstream biological processes driving ER+HER2− breast cancer cell growth, opening a new avenue for cubanyl cancer drug discovery.

Mitochondrial cross membrane potential (Δψ) alteration in MCF-7 cells were measured by monitoring Δψ sensitive TMRE fluorescence intensity. MCF-7 cells were seeded at 20,000 cells/well into a clear bottom black 96 well microplate. After treatments were added for 2 hours (FCCP control for 10 minutes), TMRE was added to a final concentration of 400 nM and incubated for 30 minutes. Media was removed and cells washed twice with PBS and 100 μL of PBS was added in the end. TMRE fluorescence was then measured with a plate reader. Results are reported as mean fluorescence intensity±S.D. (n=8, * indicates statistically significant difference from the DMSO control).

### Materials and Methods for Example B

Cell lines, Chemicals and Reagents. The MCF-7 cell line is a gift from Dr. Harikrishna Nakshatri (Indiana University) and short tandem repeat profiling (STR) verified by his laboratory.

MEM (low glucose; 1 g/L) and fetal bovine serum (FBS) were purchased from GIBCO/Invitrogen (Carlsbad, CA). Phenomenex Luna C18 (250×4.6 mm, 5-μm particle size) columns were purchased from Phenomenex (Torrance, CA). Insect cell microsomes expressing recombinant human P450 CYP3A4 and CYP2C8 (Supersomes™) were purchased from Corning (Corning, NY). Seahorse XFe analyzer assay sensor cartridges, cell culture plates, calibrant and assay medium were purchased from Agilent (Santa Clara, CA). Eicosanoid mass spectrometry standards (±)-8,9-(5Z,11Z,14Z)-EET, (±)-11,12-(5Z,8Z,14Z)-EET, (±)-14,15-(5Z, 8Z, 11Z)-EET and arachidonic acid were purchased from Cayman Chemical Co. (Ann Arbor, MI). Methylene chloride, NADPH sodium salt, EDTA, HPLC-grade acetonitrile were purchased from MilliporeSigma (St. Louis, MO). TMRE-Mitochondrial Membrane Potential Assay Kit was purchased from Abcam (Cambridge, MA).

Phospho-AMPKα (Thr172) (D79.5E) rabbit mAb #4188 (recognizes α1 and α2 subunits) and AMPKα antibody rabbit polyclonal antibody #2532 were purchased from Cell Signaling Technology, Inc. (Danvers, MA).

3-(4,5-Dimethylthiazol-2-yl)-2,5-diphenyltetrazolium Bromide (MTT) Cell Proliferation Assay. To cells grown in 96-well-tissue culture plate, 20 μl/well of MTT solution (5 mg/ml in PBS) was added. After 1-2 hours incubation at 37° C., the plate was centrifuged. Supernatant was discarded and 100 μl DMSO was added to dissolve water insoluble formazan, and the absorbance was read at 540 nm with a plate reader.

Recombinant Microsomal CYP-mediated EET Biosynthesis. CYP Supersomes™ (Corning, NY) (1 μM, final concentration) were incubated at 37° C. for 30 min in the presence of AA (120 μM) in 50 μl potassium phosphate buffer, (50 mM, pH 7.4), containing 1 mM EDTA and 1 mM NADPH. Reactions were terminated by addition of 50 μL of acetonitrile spiked with ¹³C-EET internal standards followed by vigorous vortexing. After centrifugation at 16,000×rpm for 10 minutes to precipitate proteins and other insoluble debris and 80 μL supernatant was transferred into HPLC vials and analyzed by with an LC-ESI/MRM/MS method.

LC-ESI/MRM/MS Method for EET Measurement. EETs were measured by an LC-ESI/MRM/MS analytical method on a ThermoFisher Vantage triple quadrupole mass spectrometer (ThermoFisher, San Jose, CA) coupled with an UltiMate 3000 HPLC (ThermoFisher, San Jose, CA), using argon as the collision gas. Negative ion monitoring was performed at the following diagnostic product ions: 319 m/z→155 m/z for 8,9-EET; 319 m/z→179 m/z for 11,12-EET; 319 m/z→219 m/z for 14,15-EET; 339 m/z→163 m/z for 8,9-[¹³C-20]EET; 339 m/z→233 m/z for 11,12-[¹³C-20]EET; and 339 m/z→259 m/z for 14,15-[¹³C-20]EET. Base-line resolution of EET regioisomers was achieved on a Phenomenex Luna C18 (2) reverse phase capillary column (250×0.5 mm, 5-μm particles) using the following mobile phase combinations: isocratic 5% B for 5 min, gradient 5-70% B for 5 min, hold at 70% for 30 min and then 95% for 10 min; A: 0.01% acetic acid in water, B: 0.01% acetic acid in acetonitrile; 10 μl/min flow rate. A standard curve was obtained by linear regression of the peak area ratio of authentic EET regioisomers against internal standards. The amount of EETs in samples was calculated according to the standard curve.

Measurement of oxygen consumption rate (OCR). Cells were maintained in growth medium consisting of 10% FBS at 37° C. with 5% CO2 and seeded at 25,000 cells per well in a 96-well XF96 cell culture microplates (Agilent, Santa Clara, CA). An 96 well XF sensor cartridge was hydrated and equilibrated with 200 ul calibrant in each well at 37° C. without CO₂ overnight. Growth media was replaced with 150 μL DMEM based assay media (a non-buffered medium including 2 mM L-glutamine but no sodium bicarbonate (buffering agent), glucose, or sodium pyruvate, Agilent, Santa Clara, CA) the next day and cells were incubated CO₂ free for 1 hour before the measurement of OCR by a Seahorse XFe Extracellular Flux Analyzer (Agilent, Santa Clara, CA), which measures uptake and excretion of metabolic end products in real time. After baseline measurements, 20 μl assay medium containing control vehicle or drugs were injected into corresponding wells. OCRs were measured periodically and can be reported in pmoles/minute or normalized against baseline measurements before treatment.

TMRE Δψm measurement. MCF-7 cells were plated in black wall clear bottom 96-well tissue culture plates at 20,000 cells/well and grown overnight. In addition to treatment and no treatment control groups, a non-mitochondrial TMRE staining group was included as background fluorescence control, to which 20 μM of FCCP was added and incubated for 30 minutes at 37° C. TMRE was then added to all wells at 400 nM. After 30 min incubation at 37° C., media was removed and cells were washed twice with PBS (0.2% BSA) and 100 μl of PBS (0.2% BSA) was added. Fluorescence was read at Ex 549 nm/Em 575 nm. TMRE fluorescence due to mitochondrial potential was calculated by subtracting the background fluorescence (FCCP group) from total fluorescence.

Derivation process of LR-FR- and LR-FR-PR resistant MCF7AC1 cells. The letrozole-resistant MCF7AC1 cell line (LR-MCF7AC1) was generated in vivo from the aromatase-expressing MCF7A C1 tumor-bearing athymic female nude mice that received letrozole therapy (10 μg/day, five days a week) over a period of 27 weeks. To facilitate tumor growth mice also received the estrogen precursor androstenedione (100 μg/day, five days a week), which is intratumorally converted by aromatase into estradiol (Yue W, et al. Cancer Res. 1994; 54(19):5092-5). With the emergence of the letrozole resistant tumors, these tumors were harvested from in vivo and the LR-MCF7AC1 cell line was established in vitro (Jayaraman S., et al. Breast Cancer Res. 2020; 22(1):51), where the cells grew in phenol-red free IMEM+10% FBS media containing antibiotics.

In a second in vivo study, the LR-MCF7AC1 tumor-bearing athymic female nude mice were randomized to multiple endocrine therapies including fulvestrant therapy (1000 μg/day, five days a week). One mouse harboring the LR-MCF7AC1 tumor began to develop resistance to fulvestrant therapy at 31 weeks of treatment. This fulvestrant-resistant LR-MCF7AC1 tumor was harvested from mice and reimplanted into mice for two successive generations. The 2^{nd} generation tumor was harvested from in vivo and the LR,FR-MCF7AC1 cell line was established in vitro, where the cells grew in in phenol-red free IMEM+10% FBS media containing antibiotics.

For the development of palbocilib resistant cells, LR,FR-MCF7AC1 cells were trypsinized and homogenized into single cell suspensions at a concentration of 5 cells/ml in a 96-well plate. These cells were maintained in the presence of 100 nM palbociclib in culture for a month and monitored for the growth of resistant clone. Upon development of resistant clone, these cells were transferred to a 6-well plate and expanded in the presence of the 100 nM palbociclib. Further selection was done by increasing the concentration of palbociclib to 1 mM for additional 4 weeks in phenol-red DMEM/F12+10% FBS media containing antibiotics.

Cell proliferation assay: The cells mentioned in this study were seeded in 96 wells-plates (2000-4000 cells/well) and treated 24 hours later with DMSO (vehicle control), HCB or HBB drugs at the indicated concentrations. 72 hours later, the cells were fixed in situ with 25% (v/v) glutaraldehyde, followed by staining with 0.52% crystal violet (CV) in 25% methanol (Fisher Chemical). The CV stained plates were solubilized in 100 mM sodium citrate in 50% ethanol solution and absorbance of the stain measured at 550 nm using a plate reader. Each data point represents mean±SD, obtained from six wells per treatment performed in biological triplicates. All reagents were from Sigma-Aldrich unless otherwise stated.

Resistance profiles: The letrozole- and fulvestrant-resistant MCF7AC1 (LR,FR-MCF7AC1) cells are resistant to letrozole and fulvestrant cotreatments and are capable of growth in the presence in 1 μM concentration of each drug in phenol-red DMEM/F12+10% FBS media containing antibiotics.

The letrozole-, fulvestrant- and palbociclib-resistant MCF7AC1 (LR,FR,PR-MCF7AC1) cells are resistant to letrozole, fulvestrant and palbociclib cotreatments and are capable of growth in the presence in 1 μM concentration of each drug in phenol-red DMEM/F12+10% FBS media containing antibiotics.

HCB is more potent that HBB for inhibition of letrozole resistant breast cancer cell lines letrozole, fulvestrant, and palbociclib resistant cell lines. For example, the LR/FR MCF7A1C cells exhibit an HCB IC₅₀ of 1.3 uM compared to an HBB IC₅₀ of 3.9 uM and LR/FR/PR MCF7A1C cells exhibit an HCB IC₅₀ of 1.7 uM compared to an HBB IC₅₀ of 4.9 uM. This is an ˜3 fold increase is sensitivity. MCF10A cells exhibit an HCB IC₅₀ of 2.5 uM compared to an HBB IC50 of 5.2 uM. These data show the greater clinical utility of HCB vs. HBB in the treatment of multiply resistant ER+HER2− breast cancer cells, which are aromatase inhibitor resistant, fulvestrant resistant, and cyclin dependent kinase inhibitor resistant.

### Example C. Effect of Hexyl-Cubanyl-Biguanide (HCB) and Hexyl-Benzyl-Biguanide (HBB) on SARS-CoV-2 Replication

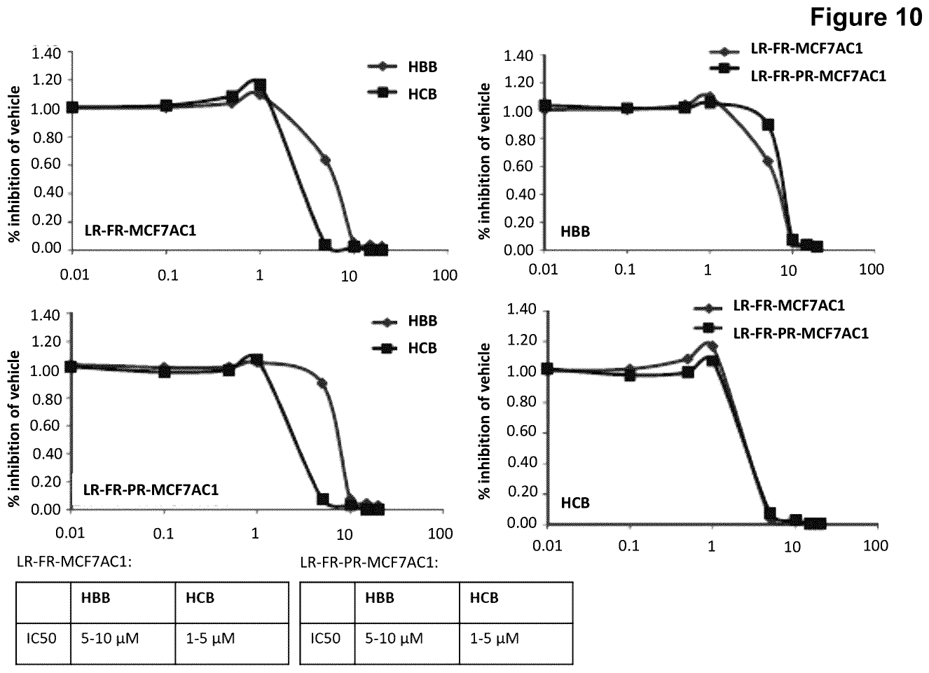

To test whether HBB or HCB exhibits antiviral activity, two assays were used. In the first assay, show in in FIG. 11, in Vero cells (African green monkey kidney cells), remdesivir was compared with HCB and HBB. In the control experiment, remdesivir showed an antiviral IC50 of 1.037 μM and an IC90 of 2.253 μM. In the cytotoxicity assay, the CC10 was >0.286 μM and the CC50 was >20 μM. The experiment testing HCB showed an antiviral IC50 of 15.629 μM and an IC90 of 19.644 μM. In the cytotoxicity assay the CC10 was 0.201 and the CC50 was 13.278 μM. The experiment testing HBB showed an antiviral IC50>20 μM and an IC90>20 μM. In the cytotoxicity assay, the CC10 was 0.091 μM and the CC50 was >20 μM.

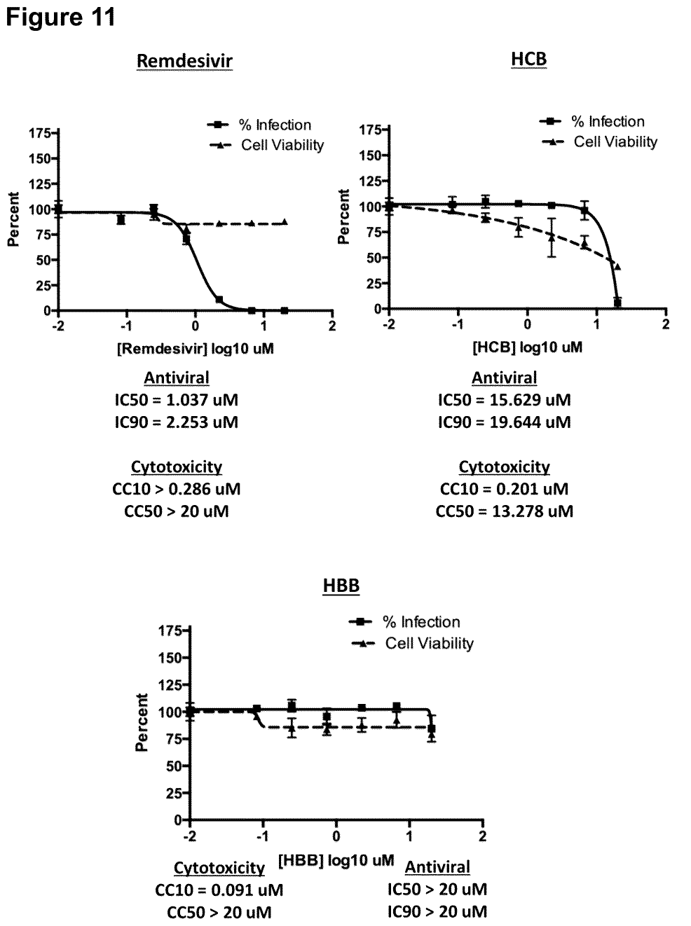

The second type of assay, shown in FIG. 12, was performed in 293T human embryonic kidney cells over-expressing the ACE2 receptor for SARS-CoV-2. In the 293T/ACE2 assay, remdesivir was compared with HCB and HBB. Remdesivir showed an antiviral IC50 of 0.02 μM an IC90 of 0.022 μM. In the cytotoxicity assay, the CC10 and CC50 values were both >0.1 μM. The experiment testing HCB showed an antiviral IC50 of 0.7 μM and an IC90 of 4.8 μM. The CC10 was 1.04 μM and the CC50 7.2 μM. The experiment testing HBB showed an antiviral IC50 of 0.82 μM and an IC90 of 11.3 μM. In the cytotoxicity assay, the CC10 was 1.5 μM and the CC50 26 μM.

Discussion. In the Vero cell assay, HCB showed much more potent activity than HBB. In the 293/T cell assay, HCB showed more potent activity than HBB, in terms of the IC90 value, which was less than half that of HBB (4.8 vs. 11.3 uM). Overall, the assays show potential for biguanide compounds, such as HCB and HBB, as antivirals.

### Materials and Methods for Example C

Cell lines. HEK293T/17 expressing human ACE2 were cultured in Dulbecco's Modified Eagle's Medium (Corning) supplemented with 10% Fetal Bovine Serum (Gibco, Life Technologies) and 1% Penicillin-Streptomycin (Corning) and maintained at 37° C. in a humidified atmosphere of 5% CO2. HEK-293T/17 parental cells were procured from the UCSF Cell Culture Facility. STR analysis by the Berkeley Cell Culture Facility authenticates HEK-293T/17 cells with 94% probability. Cells were tested using the MycoAlert™ Mycoplasma Detection Kit (Lonza LT07-318) and were negative: B/A ratio<1 (no detected mycoplasma).

Vero E6 cells were purchased from the ATCC VERO C1008 [Vero 76, clone E6, Vero E6] (ATCC® CRL-1586™)

Viral growth and cytotoxicity assays in the presence of inhibitors. 2,000 Vero E6 cells were seeded into 96-well plates in DMEM (10% FBS) and incubated for 24 h at 37 C, 5% CO2. Vero E6 cells used were purchased from ATCC and thus authenticated (VERO C1008 [Vero 76, clone E6, Vero E6] (ATCC® CRL-1586™); tested negative for mycoplasma contamination prior to commencement). Two hours before infection, the medium was replaced with 100 μL of DMEM (2% FBS) containing the compound of interest at concentrations 50% greater than those indicated, including a DMSO control. The Vero E6 cell line used in this study is a kidney cell line; therefore, we cannot exclude that lung cells yield different results for some inhibitors (also see Methods Institut Pasteur). Plates were then transferred into the BSL3 facility and 100 PFU (MOI 0.025) was added in 50 μL of DMEM (2% FBS), bringing the final compound concentration to those indicated. Plates were then incubated for 48 h at 37 C. After infection, supernatants were removed and cells were fixed with 4% formaldehyde for 24 hours prior to being removed from the BSL3 facility. The cells were then immunostained for the viral NP protein (anti-sera produced in the Garcia-Sastre lab; 1:10,000) with a DAPI counterstain. Infected cells (488 nM) and total cells (DAPI) were quantified using the Celigo (Nexcelcom) imaging cytometer. Infectivity is measured by the accumulation of viral NP protein in the nucleus of the Vero E6 cells (fluorescence accumulation). Percent infection was quantified as ((Infected cells/Total cells)−Background)*100 and the DMSO control was then set to 100% infection for analysis. The IC50 and IC90 for each experiment were determined using the Prism (GraphPad Software) software.

### Example D. Pharmacokinetics

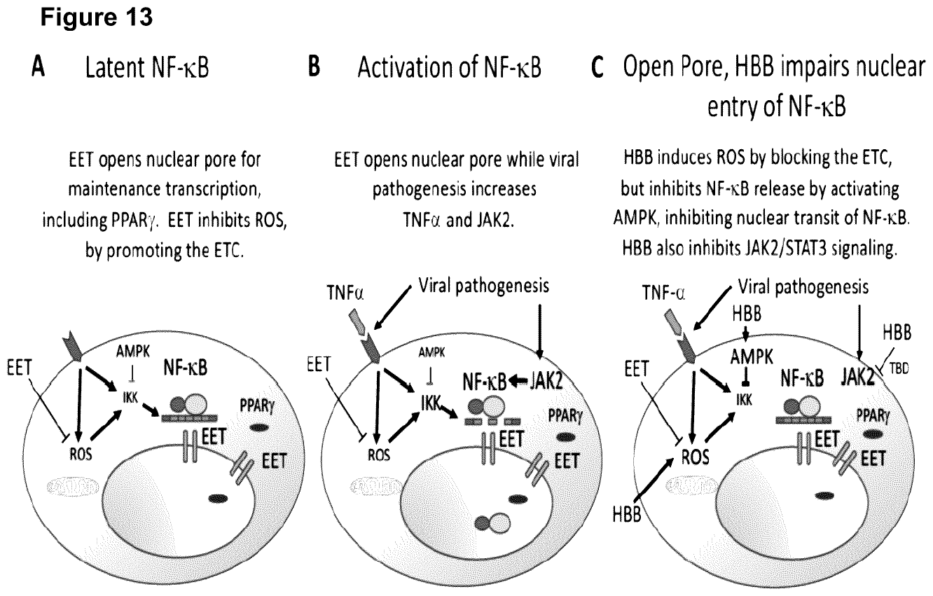

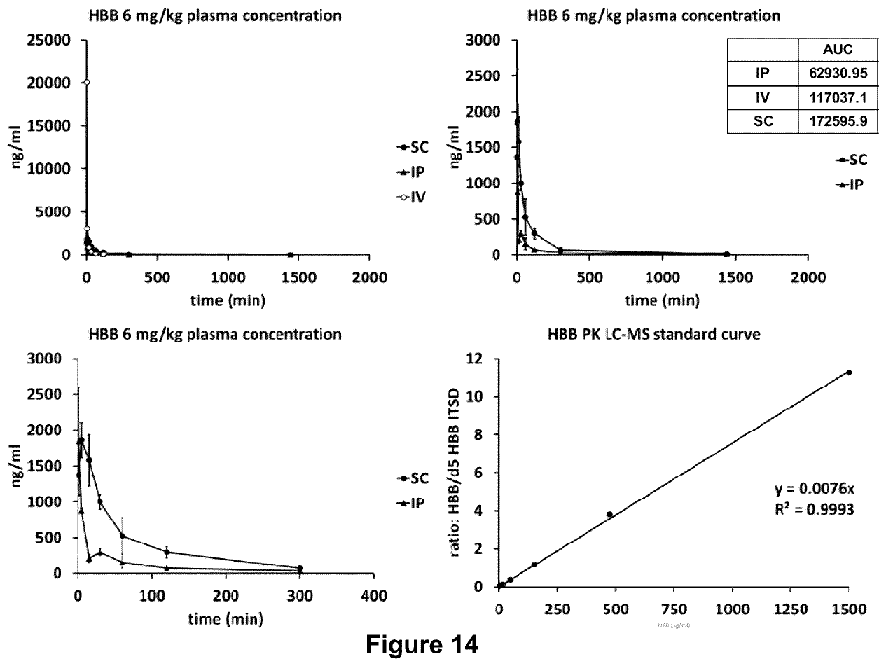

That subcutaneous administration of HBB and HCB is better than IV, intraperitoneal or oral. 4. Subcutaneous administration of HBB and HCB is the preferred method of delivery. Biguanides are usually delivered orally and subcutaneous delivery of anti-diabetic drugs is not usually done. Nonetheless we find, unexpectedly, that there is better AUC. (FIGS. 14-15) The finding that HBB and HCB are both more bioavailable indicates a class effect which is novel.

HBB PK method: HBB in mice plasma was determined with an LC-MS/MS method using d5-HBB as the internal standard.

Sample preparation: HBB in 150 μL mice plasma was extracted by addition of 300 μL acetonitrile spiked with d5-HBB internal standard (0.5 ug/ml). The extraction mixture was centrifuged at 12,000×g for 5 minutes and 150 μL of supernatant was saved for LC-MS/MS analysis.

LC-MS/MS method: A Quattro Ultima mass spectrometer (Waters, Milford, MA) equipped with an Acquity UPLC (Waters, Milford, MA) and interfaced through an MassLynx software (Waters, Milford, MA) was used. HBB and d5-HBB were resolved at base line with a Zorbax Eclipse XDG-C18 column (4.6×50 mm, 1.8 micron) (Agilent, Santa Clara, CA). Mobile phases were A: water (0.1% formic acid) and B: acetonitrile (0.1% formic acid). The flow rate was 250 μL/min. Elution gradient was as follow: 0-5 min, 90% A and 10% B; 5 min, 10% A and 90% B; 5.25 min, 90% A and 10% B; 10 min, 90% A and 10% B. At positive mode, ion transition for HBB 276.01 mz to 91.02 mz and d5-HBB 281.09 to 96.06 were monitored.

Quantification: Quantitation was carried out by the MassLynx software based on an HBB standard curve (1.5-1500 ng/ml). Per FDA bioanalytical method validation guideline, standards and plasma samples were bracketed with QC samples to ensure accuracy and precision of the measurement.

HCB PK method: HCB in mouse plasma was determined with an LC-MS/MS method using d5-HBB as the internal standard.

Sample preparation: HCB in 150 μL mice plasma was extracted by addition of 300 μL acetonitrile spiked with d5-HBB internal standard (0.5 ug/ml). The extraction mixture was centrifuged at 12,000×g for 5 minutes and 150 μL of supernatant was saved for LC-MS/MS analysis.

LC-MS/MS method: A Quattro Ultima mass spectrometer (Waters, Milford, MA) equipped with an Acquity UPLC (Waters, Milford, MA) and interfaced through an MassLynx software (Waters, Milford, MA) was used. Neo biguanide and d5-HBB were resolved at base line with a Zorbax Eclipse XDB-C18 Rapid Resolution HT (P.M. 927975-902) column (4.6×50 mm, 1.8 micron) (Agilent, Santa Clara, CA). Mobile phases were A: water (0.1% formic acid) and B: acetonitrile (0.1% formic acid). The flow rate was 250 μL/min. Elution gradient was as follow: 0-5 min, 90% A and 10% B; 5 min, 10% A and 90% B; 5.25 min, 90% A and 10% B; 10 min, 90% A and 10% B. At positive mode, ion transition for d5-HBB 281.09 to 96.06 and corresponding neo biguanide parent daughter ion transition were monitored.

Quantification: Quantitation was carried out by the MassLynx software based on an HCB standard curve (1.5-1500 ng/ml). Per FDA bioanalytical method validation guideline, standards and plasma samples were bracketed with QC samples to ensure accuracy and precision of the measurement.

### Example E. Synthesis Methods

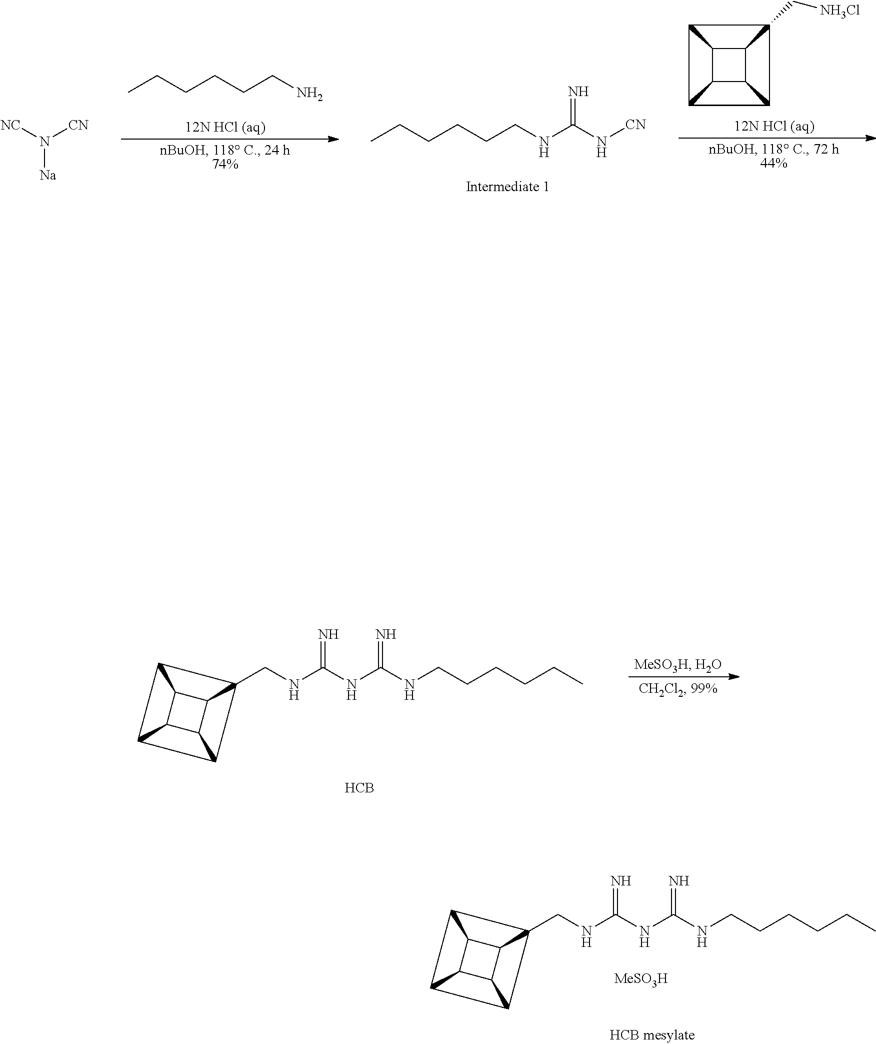

Synthesis of intermediate 1 for HCB (Hexyl Cubanomethyl Biguanide): Sodium dicyanamide (7 g, 78.28 mmol, 1.1 equiv) was added to hexylamine (7.2 g, 71.16 mmol) in nBuOH (60 mL). The reaction mixture was stirred in ice bath for 5 min and 12 N HCl (aq) (6.5 mL, 78.28 mmol, 1.1 equiv) was added dropwise in ice bath. The reaction mixture was stirred at ambient temperature for 10 min, and then heated and stirred under reflux for 24 h. After 24 h, the solvent was removed under reduced pressure to dryness. The crude product was suspended in water and ethyl acetate and filtered and rinsed with water. The product obtained from the filtration was used for the next step without further purification (8.823 g, 74% yield): ¹H NMR (400 MHz, DMSO-d₆) δ 6.67 (d, J=69.5 Hz, 3H), 3.09-2.95 (m, 2H), 1.47-1.34 (m, 2H), 1.33-1.17 (m, 6H), 0.87 (t, J=6.8 Hz, 3H).

Synthesis of HCB (Hexyl Cubanomethyl Biguanide): KH3P-105 (101.7 mg, 0.604 mmol) was added to cubanomethyl amine hydrochloride (201.6 mg, 1.188 mmol, 1.97 equiv) solution in nBuOH (6 mL) and the reaction mixture was stirred under reflux for 3 days. After 3 days, the solvent was removed under reduced pressure to dryness. The crude product was suspended in 1N HCl (20 mL) and ethyl acetate (20 mL). The aqueous layer with an insoluble substance was separated and the product was obtained from the aqueous layer by filtration and rinse with water (80.1 mg, 44% yield): ¹H NMR (400 MHz, DMSO-₆) δ 4.06-3.96 (m, 1H), 3.88 (q, J=3.3, 2.4 Hz, 6H), 3.38 (d, J=5.2 Hz, 2H), 3.10 (d, J=6.8 Hz, 2H), 1.46 (s, 2H), 1.27 (s, 6H), 0.92-0.82 (m, 3H).

Synthesis of HCB-mesylate: Methanesulfonic acid (2.66 mL, 0.1 M in H₂O) in water was added to HCB (80.1 mg, 0.266 mmol) in DCM (10 mL) and stirred at rt. The solvent was removed under reduced pressure. The product was obtained as a solid (105 mg, 99%): ¹H NMR (400 MHz, DMSO-d₆) δ 4.00 (dt, J=4.9, 2.4 Hz, 1H), 3.88 (dt, J=7.1, 2.2 Hz, 6H), 3.38 (d, J=5.3 Hz, 2H), 3.16-3.04 (m, 2H), 2.32 (s, 3H), 1.46 (s, 2H), 1.27 (q, J=3.3 Hz, 6H), 0.90-0.81 (m, 3H).

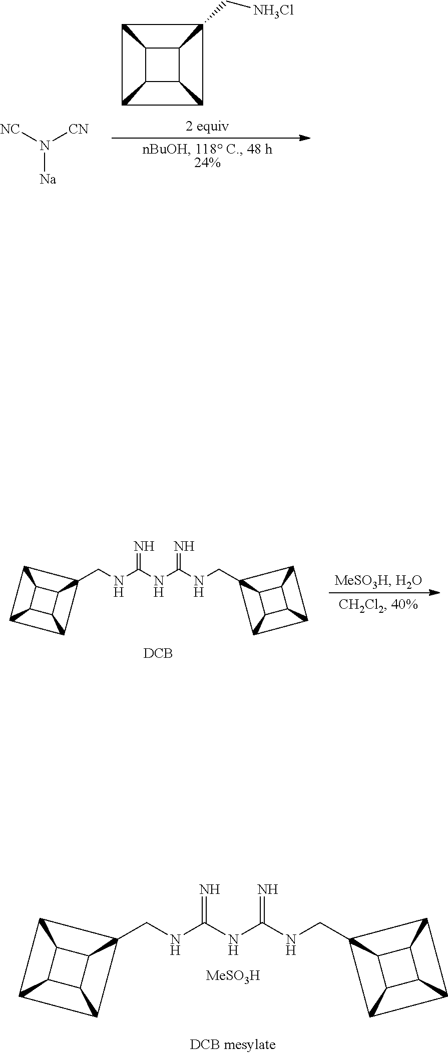

Synthesis of DCB (Dicubanomethyl Biguanide): Sodium dicyanamide (23.9 mg, 0.265 mmol) was added to cubanomethylamine hydrochloride (100 mg, 0.589 mmol, 2.2 equiv) in nBuOH (5 mL). The reaction mixture was heated and stirred under reflux for 48 h. After 48 h, the solvent was removed under reduced pressure to dryness. The crude product was suspended in water and ethyl acetate and filtered and rinsed with water. The product obtained from the filtration was used for the next step without further purification (23.1 mg, 24%): ¹H NMR (400 MHz, DMSO-d₆) δ 4.00 (qt, J=4.7, 2.4 Hz, 2H), 3.85 (dddd, J=16.6, 6.5, 4.7, 2.8 Hz, 12H), 3.36 (d, J=5.7 Hz, 2H), 3.30 (d, J=5.8 Hz, 2H).

### Synthesis of DCB-Mesylate

Methanesulfonic acid (650 μL, 0.1 M in H₂O, 1 equiv) in water was added to HCB (21.8 mg, 0.065 mmol) in DCM (10 mL) and stirred at rt. The solvent was removed under reduced pressure. The product was obtained as a solid (11 mg, 40%): ¹H NMR (400 MHz, DMSO-d₆) δ 4.01 (d, J=5.1 Hz, 2H), 3.94-3.80 (m, 12H), 3.41-3.27 (m, 4H), 2.33 (s, 3H).

### Synthesis of Fluorinated Compounds

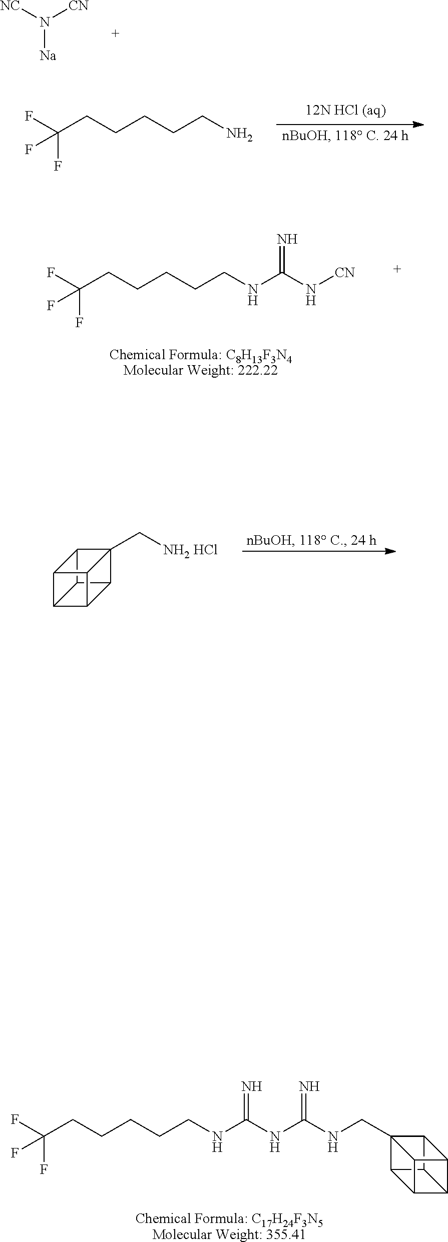

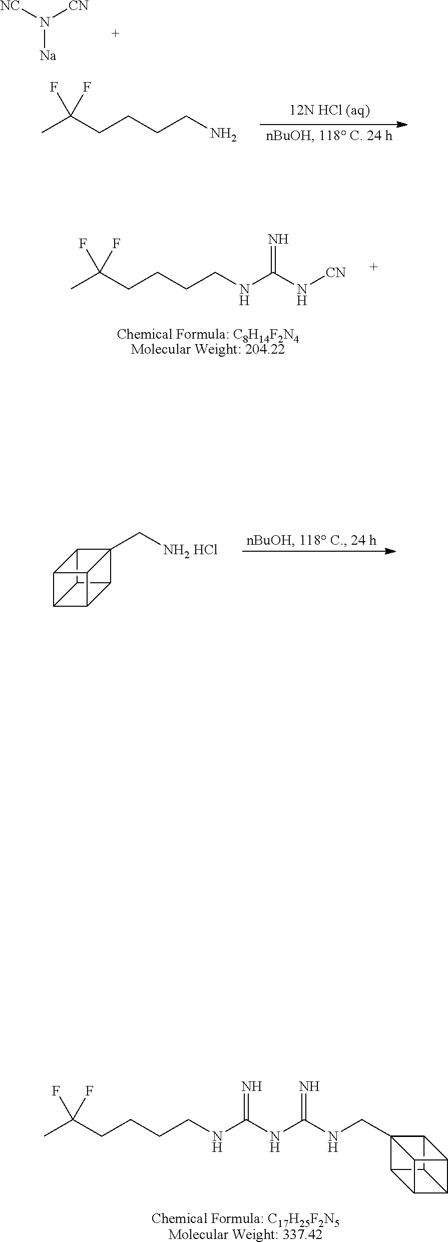

Halo-substituted, cubanyl compounds may be prepared similarly to those described for HCB. Briefly, sodium dicyanamide may be added to a halo-substituted alkylamine (e.g., 6,6,6-trifluor-hexylamine or 5,5-difluoro-hexylamine) in nBuOH. The reaction mixture may be stirred in ice bath for 5 min and 12 N HCl (aq) was added dropwise in ice bath. The reaction mixture may be stirred at ambient temperature for 10 min, and then heated and stirred under reflux for 24 h. After 24 h, the solvent may be removed under reduced pressure to dryness.

N1-6′,6′,6′-trifluorohexyl-N5-(cuban-1′-yl)methyl-biguanide (HCB-F3 mesylate) synthesis. 6,6,6-trifluorohexylamine (0.48 ml, 3.06 mmol) was mixed with 8 ml of n-butanol and 0.27 ml of concentrated HCl before adding sodium dicyanamide (0.4 g, 4.5 mmol). The solution was refluxed at 118° C. for 24 h followed by evaporation of n-butanol to yield a sticky yellow residue, which was taken up in dichloromethane, washed with water, and extracted with 3×CH₂Cl₂. The combined organics were dried over Na₂SO₄, filtered, and concentrated under reduced pressure to a yellow oil that was confirmed by NMR to be intermediate 6′,6′,6′-trifluorohexyl-3-cyanoguanidine, which was used in the next step without further purification. NMR data for structure verification of 6′,6′,6′-trifluorohexyl-3-cyanoguanidine: 1H NMR (400 MHz, DMSO-d₆) δ 7.2-6.6 (br m, 3H), 3.03 (q, J=6.59 Hz, 2H), 2.23 (m, 2H), 1.45 (m, 6H). Next, 376 mg of 6′,6′,6′-trifluorohexyl-3-cyanoguanidine was dissolved in 10 ml of n-butanol followed by addition of 350 mg of (cuban-1-yl)methylamine hydrochloride. The reaction mixture was refluxed at 118° C. for 24 h before distilling off the n-butanol and concentrating the remaining residue to a solid under reduced pressure. The solid was dissolved in 10 ml 1N HCl followed by addition of 10 ml of ethyl acetate. Brown colored substance precipitated in the aqueous phase and was filtered out and washed with water and ethyl acetate to yield N1-6′,6′,6′-trifluorohexyl-N5-(cuban-1-yl)methyl-biguanide. N1-6′,6′,6′-trifluorohexyl-N5-(cuban-1-yl)methyl-biguanide 1H NMR (400 MHz, DMSO-d₆) δ 4.1-4.8 (br s, 5H), 4.0 (br m, 1H), 3.9 (br m, 6H), 3.38 (d, J=4.4 Hz, 2H), 3.10 (br m, 2H), 2.22 (br m, 2H), 1.37 (br m, 6H).

N1-5′,5′-difluorohexyl-N5-(cuban-1-yl)methyl-biguanide (HCB-2F mesylate) synthesis. 5,5-difluorohexylamine (250 mg, 1.82 mmol) was mixed with 10 ml of n-butanol and 0.17 ml of concentrated HCl before adding sodium dicyanamide (0.32 g, 3.6 mmol). The solution was refluxed at 118° C. for 24 h followed by evaporation of n-butanol to yield a light-yellow oil, which was taken up in ethyl acetate, washed with water, and extracted with 3× ethyl acetate. The combined organics were concentrated under reduced pressure to a white wax, which was used in the next step without further purification. Next, 607 mg of presumed 5′,5′-difluorohexyl-3-cyanoguanidine was dissolved in 10 ml of n-butanol followed by addition of 509 mg of (cuban-1-yl)methylamine hydrochloride. The reaction mixture was refluxed at 118° C. for 24 h before distilling off the n-butanol and concentrating the remaining residue to a solid under reduced pressure. The solid was dissolved in 10 ml 1N HCl followed by addition of 10 ml of ethyl acetate. The aqueous phase was collected, and 10 ml ethyl acetate was added again. White substance precipitated the next morning, which was filtered out and washed with water and ethyl acetate to yield N1-5′,5′-difluorohexyl-N5-(cuban-1-yl)methyl-biguanide. N1-5′,5′-difluorohexyl-N5-(cuban-1-yl)methyl-biguanide 1H NMR (400 MHz, DMSO-d₆) δ 4.1-4.8 (br s, 5H), 4.01 (br m, 1H), 3.87 (br m, 6H), 3.37 (d, J=5.32 Hz, 2H), 3.12 (br m, 2H), 1.87 (br m, 2H), 1.59 (t, J=18.9 Hz, 3H), 1.47 (br m, 4H).

### Example F. HCB Binds to CYP3A4 by Soret Band Spin Shift Analysis

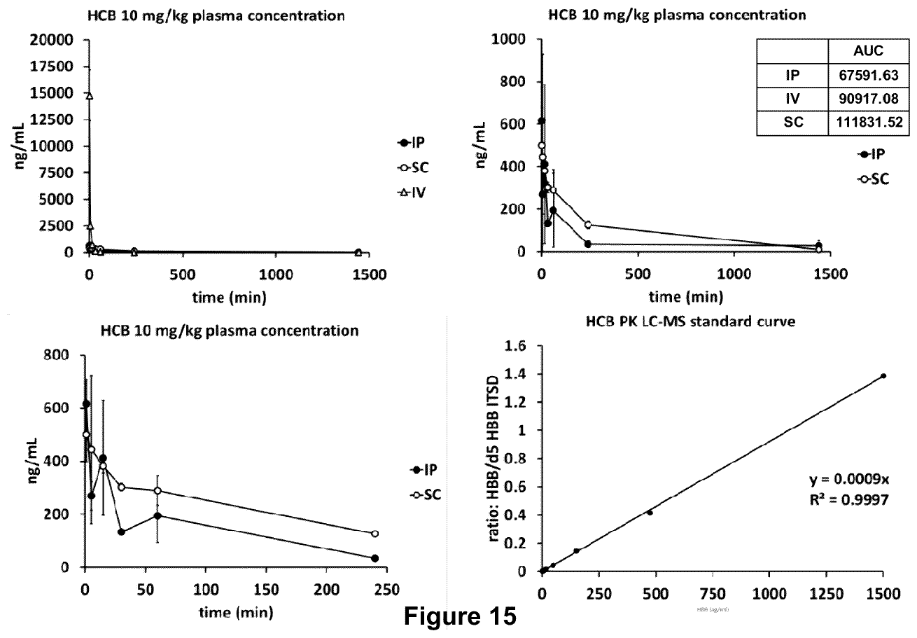

HCB induces a low-to-high spin transition in 3A4, meaning that it binds within the active site near the heme (FIG. 16). This result shows direct interaction between HCB and the CYP3A4 target. The spin shift is partial and the estimated Kd is quite low, 74 uM. In contrast, HBB has a published Ks, which estimates Kd, of 164 uM¹. This indicates that HCB has significantly higher affinity for the CYP3A4 target.

A. HBB binds soluble CYP3A4 Δ3-22, dependent on the heme iron. The HBB binding spectrum is shown in red. Control spectra with dithionite reduction (blue) and CO adduct formation (green) are shown as well as untreated CYP3A4 Δ3-22 (black). B. HBB docked to truncated CYP3A4 Δ3-22 induces a low to high spin shift (left) with a Ks=74±7 μM (right). (FIG. 16)

### Example G. HCB Binding and Inhibition of Midazolam Hydroxylation by CYP3A4 in Nanodiscs

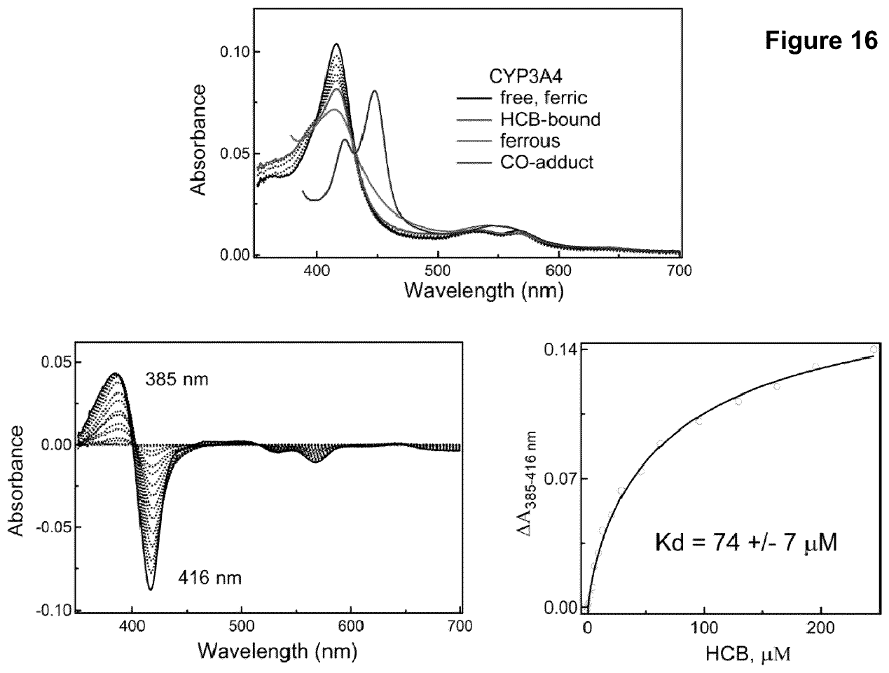

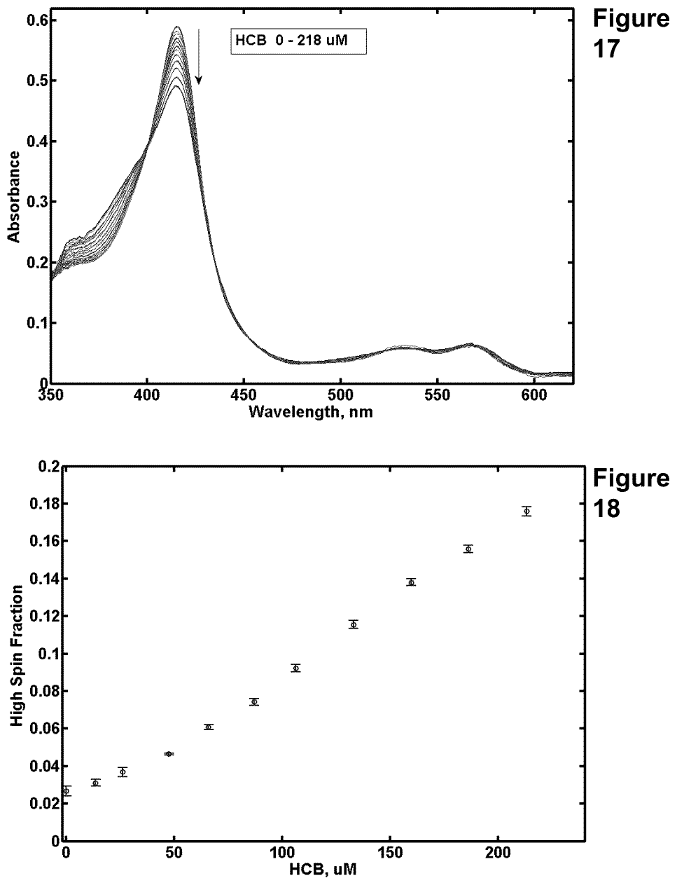

Titration of ˜6 uM CYP3A4 in POPC Nanodiscs was done using 16 mM stock solution obtained by 6-fold dilution of the original 100 mM DMSO solution in methanol. Aliquots of 0.1-0.2 μL were added to 120 μL solution of CYP3A4-ND in semi-micro cell (10 mm pathlength). Spectra with background corrected for scattering are shown in FIG. 17, and titration data points in FIG. 18.

Based on these results, we suggest that there may be two binding sites, the first binding with higher affinity happens with virtually no spin shift, i.e. HCB is not sufficiently close to the heme iron. At much higher concentrations the second binding with lower affinity results in partial spin shift. We did not reach saturation because of increasing turbidity at HCB concentrations higher than 250 uM. (If needed, we can try again and get spectral measurements at higher concentrations of HCB).

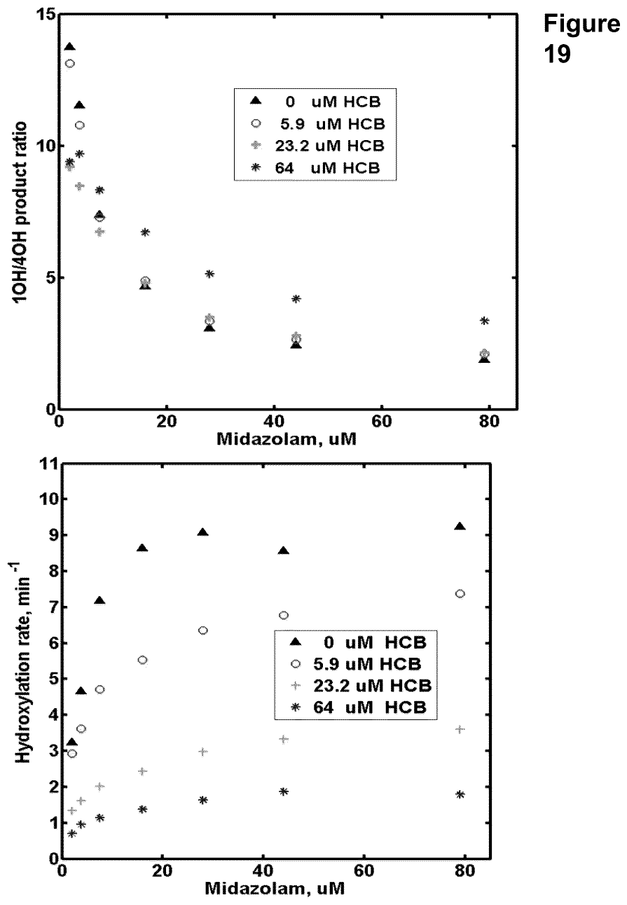

In order to obtain more information on the binding modes and inhibiting effects of HCB, we compared midazolam (MDZ) hydroxylation in the absence of HCB and in the presence of 5.9 uM, 23.2 uM and 64 uM HCB. These concentrations were selected based on IC50 for arachidonic acid metabolism mentioned in the first e-mail (low HCB concentration) and on our titration results. We used seven MDZ concentrations and measured both 1 OH and 4 OH hydroxy-MDZ products using HPLC with absorbance detection at 240 nm. Results are shown below in FIG. 19.

Significant inhibition of MDZ hydroxylation is observed already at 5.9 uM HCB, and even stronger effect is seen at higher concentrations. Interestingly, almost no effect on the 1 OH/4 OH ratio is observed at low HCB concentrations, but clear and reproducible effect is seen at 64 uM HCB. This means that HCB is not only an inhibitor, but also an allosteric effector for CYP3A4 as probed using MDZ hydroxylation.

Overall, spin shift titration results and MDZ hydroxylation in the presence of HCB may be tentatively explained as follows. The first HCB molecule binds inside the substrate binding pocket and inhibits MDZ hydroxylation (and other substrates, as mentioned in the first e-mail), however with virtually no spin shift. At higher concentrations the second HCB molecule binds at the second low-affinity site (possibly not saturating it at concentrations below 220 uM under our experimental conditions with CYP3A4-ND). This second binding perturbs position of the first HCB molecule so that the high spin signal is observed. The same second binding event at high HCB concentration also changes the 1 OH/4 OH ratio in MDZ hydroxylation reaction, while this ratio is not changing at low HCB concentrations, when this low-affinity second site is not populated.

### Example H. Toxicity

HBB is less toxic and equally effective in terms of tumor control when given by the subcutaneous route at the MTD of 12/mg/kg/day. Under these conditions we inhibit oxidative phosphorylation in ER+/HER2− MCF-7 tumors. Subcutaneous HBB has 2.6-fold greater AUC than ip.

| | HL_Lambda_z | Cmax | Tmax | AUC INF_obs |
|---|---|---|---|---|
| ROUTE | (hr) | (ng/mL) | (min) | (min*ng/mL) |
| IP | 8.23 | 1842.80 | 1.00 | 6.8 × 104 |
| IV | 7.51 | 20090.5 | 1.00 | 1.1 × 105 |
| SC | 8.13 | 1860.20 | 5.00 | 1.8 × 105 |
| | Cl_obs | Vz_obs | Cl_F_obs | Vz_F_obs |
| ROUTE | (mL/min/kg) | (mL/kg) | (mL/min/kg) | (mL/kg) |
| IP | | | 88.25 | 62842 |
| IV | 50.65 | 32933 | | |
| SC | | | 32.89 | 23131 |

Subcutaneous HBB is less toxic and MTD is higher than ip. MTD for HBB ip is 6 mg/kg 4 times per week, no more than 2 consecutive days. MTD for HBB sc is 12 mg/kg daily.

Study 1 was performed by dose escalation of SC HBB from 4 mg/kg qd to 12 mg/kg qd with female CD-1 mice (Envigo) approximately 4-8 weeks of age. N=5 mice per HBB dose group, n=10 naïve control mice.

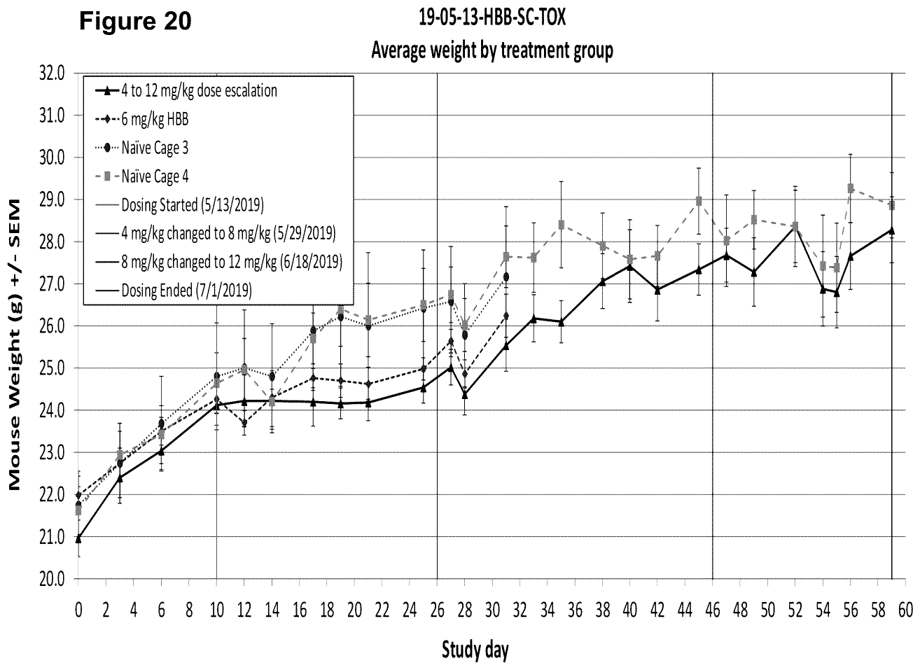

As shown in FIG. 20, dose escalation of HBB with daily SC dosing from 4 to 12 mg/kg over an approximately 2-month period is well tolerated. No significant changes in weight gain or any adverse clinical signs were observed.

Study 2 two was performed by dose escalation of SC HBB from 16 mg/kg qd to 24 mg/kg qd with female BALB/c mice (Envigo) approximately 6-8 weeks of age. N=4 mice per HBB dose group, n=3 naïve control mice

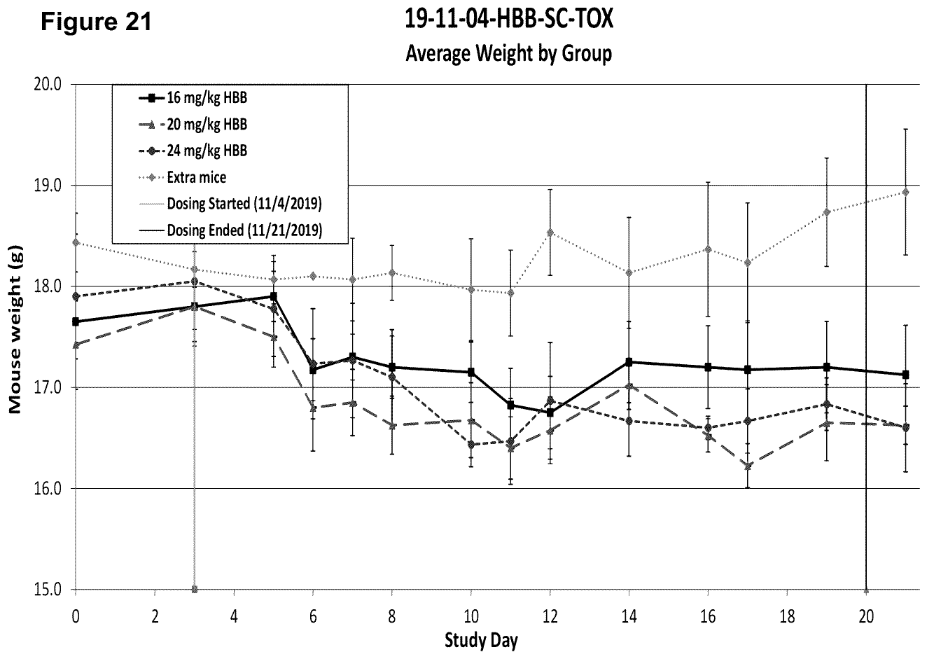

As shown in FIG. 21, daily SC dosing of HBB is not as well tolerated at doses above 12 mg/kg. There was some weight loss and skin-related toxicities were observed.

Study 3 was performed by dose escalation of SC HBB from 16 mg/kg qd to 24 mg/kg qd with female C57BL/6 mice (CR) approximately 6-8 weeks of age. N=12 for HBB dose group, n=3 naïve control mice.

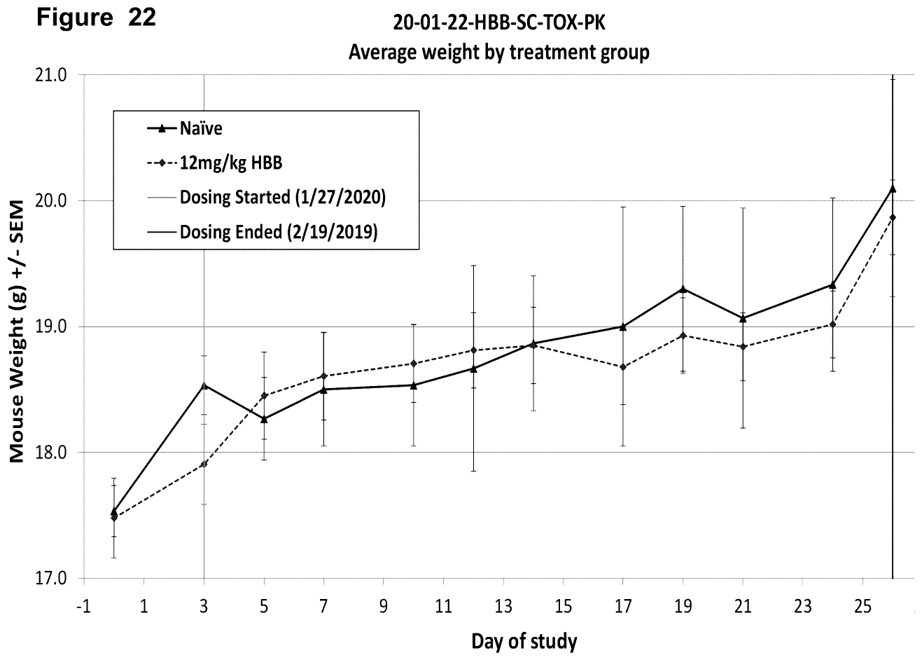

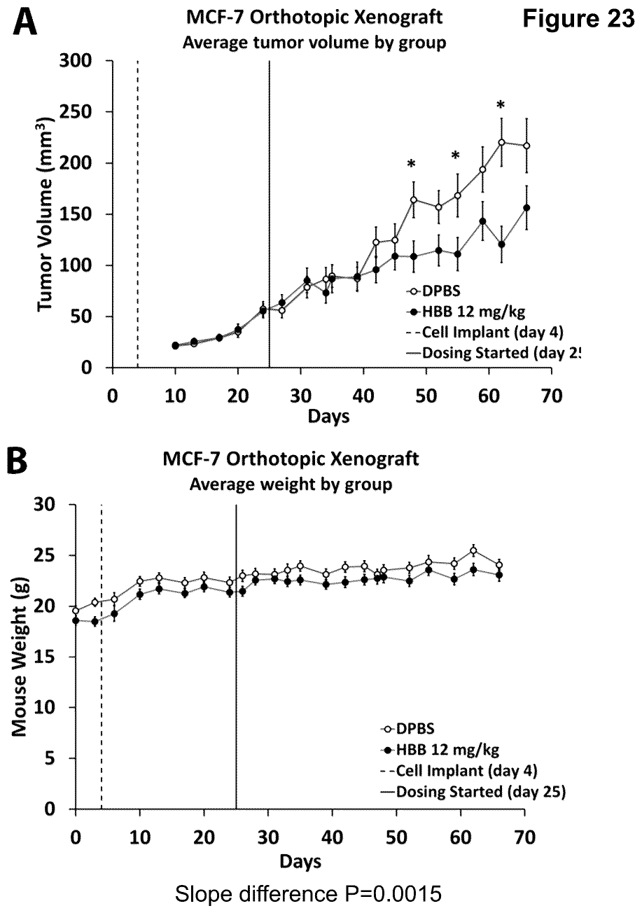

As shown in FIG. 22, daily SC dosing of HBB from study day 3 to day 26 is well tolerated. No significant changes in weight gain or any adverse clinical signs were observed. SC administration results in higher MTD and no weight loss. FIG. 23

### Example I. Microsomal Stability

Microsomal stability of HBB and HCB were evaluated in human and mouse liver microsomes. 10 μM of HBB or HCB was tested in a biological matrix comprising of human or mouse liver microsomes (1 mg/mL) (Table 6). NADPH (2mM) was added as a cofactor. Total volume was 200 μL.

TABLE 6
Liver microsomal information

| Species | Strain & Gender | Cat. No. | Lot No. | Source |
|---|---|---|---|---|
| Human | Pooled, mixed | 452117 | 38295 | Corning |
| Mouse | Male | 452701 | 00100003 | Corning |

HBB or HCB was incubated for 0, 30, or 60 min at 37° C. Incubations were quenched with 2 volumes of acetonitrile followed by centrifugation for 10 min at 16,000 g. Supernatant was analyzed by LC-MS/MS under the conditions listed in Table 7.

TABLE 7
Instrumentation & Analytical Conditions

Instrumentation
Dionex UltiMate 3000 UHPLC system (Thermo Fisher Scientific, USA)
Thermo Scientific Q Exactive (Thermo Fisher Scientific, USA)
LC conditions
Column: Waters XSelect HSS T3, 100 × 2.1 mm, 2.5 μm
Solvents: A, water (0.1% formic acid); D, acetonitrile (0.1% formic acid)
Flow rate: 500 μL/min
Program: 0-1.5 min, 5% D, 1.5-9 min, 5%-50% D, 9-12 min, 50%-100%
D, 12-14 min, 100% D, 14-14.3 min, 100%-5% D, 14.3-15 min, 5% D;
MS conditions
Ionization mode: Positive mode
Spray Voltage: 4.0 kV
Aux gas flow rate: 15
Aux gas heater temp (° C.): 350° C.
Scan type: Full MS/ddMS²
Resolution: 70,000
AGC Target: 3 × e⁶
NCE/stepped NCE: 30, 40, 50

HBB degraded via oxidation, dehydrogenation, and glucuronidation. HCB degraded via oxidation, dehydrogenation, and hydrogenation. After 60 min, HCB was more stable than HBB in both human [HCB (48.18%)/HBB (19.19%)] and mouse [HCB (18.63%)/HBB (8.95%)] exosomes as determined by parent metabolite peak area.

### Example J. Stability with Recombinant Human CYP3A4 and 2C8 Enzymes

Stability of HCB with recombinant human CYP3A4 and CYP2C8 enzymes was evaluated. 10 μM HCB was tested wich CYP3A4 and 2C8 (200 pmol/L) (Table 8). NADPH (2 mM) was added as a cofactor. Total volume was 200 μL.

TABLE 8
Recombinant human CYP enzyme product information

| CYP | Cat. No. | Lot No. | Source |
|---|---|---|---|
| 3A4 | CYP02 | C3A4R046B | CYPEX |
| 2C8 | CYP017 | C2C8R006D | CYPEX |

HCB was incubated for 0, 30, or 60 min at 37° C. Incubations were quenched with 2 volumes of acetonitrile followed by centrifugation for 10 min at 16,000 g. Supernatant was analyzed by LC-MS/MS under the conditions listed in Table 7.

HCB degraded via oxidation, dealkylation, and hydrogenation. After 60 min, parent metabolite peak area was 93.91% (CYP3A4) and 99.78% (CYP2C8).

We claim:

1. A compound of formula 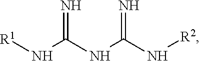
or a pharmaceutically acceptable salt thereof, wherein R¹ comprises a cubanyl-substituted alkyl and R² comprises a substituted or unsubstituted alkyl.

2. The compound of claim 1, wherein the compound is

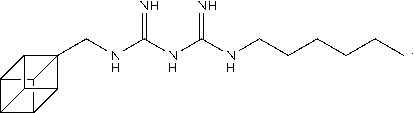 [SMILES: CCCCCCNC(=N)NC(=N)NCC12C3C4C5C3C1C5C42]

3. The compound of claim 1, wherein the compound is

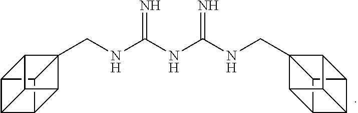 [SMILES: N=C(NCC12C3C4C5C3C1C5C42)NC(=N)NCC12C3C4C5C3C1C5C42]

4. The compound of claim 1, wherein the R² is a halo-substituted alkyl.

5. The compound of claim 4, wherein the compound is

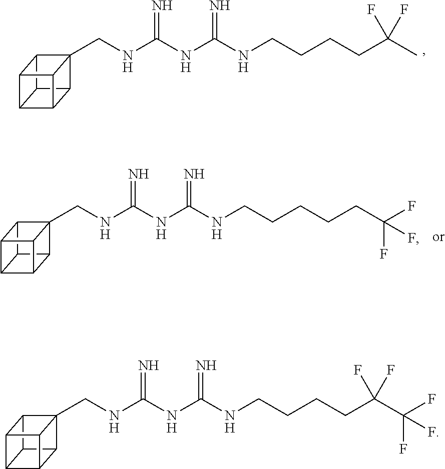 [SMILES: CC(F)(F)CCCCNC(=N)NC(=N)NCC12C3C4C5C3C1C5C42 ; N=C(NCCCCCC(F)(F)F)NC(=N)NCC12C3C4C5C3C1C5C42 ; "or" ; N=C(NCCCCC(F)(F)C(F)(F)F)NC(=N)NCC12C3C4C5C3C1C5C42]

6. The pharmaceutically acceptable salt according to any one of claims 1-5, wherein the salt is a mesylate salt.

7. A pharmaceutical composition comprising a therapeutically effective amount of the compound according to any one of claims 1-5 and a pharmaceutically acceptable excipient, diluent, or carrier.

8. The pharmaceutical composition of claim 7, wherein the pharmaceutical composition is formulated for subcutaneous administration.

9. A method for treating a subject in need of a treatment for a cancer, the method comprising administering an effective amount of the compound according to any one of claims 1-5 to the subject.

10. The method of claim 9, wherein the subject is in need of a treatment for a breast cancer.

11. The method of claim 10, wherein the subject is in need of a treatment for an ER+ breast cancer.

12. The method of claim 11, wherein the subject is in need of a treatment for a HER2+ breast cancer.

13. The method of claim 11, wherein the subject is in need of a treatment for a HER2− breast cancer.

14. The method of claim 10, wherein the subject is in need of a treatment for a HER2+ breast cancer.

15. The method of claim 14, wherein the subject is in need of a treatment for an ER− breast cancer.

16. The method of claim 10, wherein the subject is in need of a treatment for a triple negative breast cancer.

17. The method of any one of claims 9-16, wherein the compound is administered subcutaneously.

18. The method of any one of claims 9-17, wherein the compound is co-administered with an immunotherapy.

19. The method of any one of claims 9-18, wherein the cancer is a CYP-dependent tumor or a mTOR-dependent tumor.

20. The method of any one of claims 9-19, wherein the cancer is dependent on an EET for angiogenesis or nuclear signaling.

21. The method of any one of claims 9-20, wherein the cancer is dependent on oxidative phosphorylation.

22. The method of any one of claims 9-21, wherein the cancer is a therapy-resistant cancer.

23. A method for treating a subject in need of a treatment for a viral infection, the method comprising administering an effective amount of a biguanide compound to the subject.

24. The method of claim 23, wherein the viral infection is a coronavirus infection.

25. The method of claim 24, wherein the viral infection is a SARS-CoV-2 infection.

26. The method of any one of claims 23-25, wherein the biguanide compound is the compound according to any one of claims 1-5.

27. The method of any one of claims 23-26, wherein the compound is administered subcutaneously.

28. The method of any one of claims 23-27, wherein the virus is a mTOR dependent virus or an oxidative phosphorylation dependent virus.

29. The method of any one of claims 23-28 further comprising co-administering an antiviral agent.

30. A method for increasing the bioavailability of a biguanide compound, the method comprising subcutaneously administering the biguanide compound to a subject in need of the biguanide compound.

31. The method of claim 30, wherein bioavailability of the biguanide, compound as measured by the AUC, is larger when the biguanide compound is administered subcutaneously than when administered orally, intravenously, or intraperitoneally.

32. The method of any one of claims 30-31, wherein the biguanide is less toxic to the subject when the biguanide compound is administered subcutaneously than when administered orally, intravenously, or intraperitoneally.

33. The method of any one of claims 30-32, wherein the maximum tolerated dose (MTD) is larger when the biguanide compound is administered subcutaneously than when administered orally, intravenously, or intraperitoneally.

34. The method of any one of claims 30-33, wherein the biguanide compound is the compound according to any one of claims 1-5.

35. The method of any one of claims 30-34 wherein the subject is in need of a treatment for cancer.

36. The method of any one of claims 30-34, wherein the subject is in need of a treatment for a viral infection.
